SEQUENCE LISTING (13 sequences)

SEQ ID NO: 1 - DNA, 24 bp, synthetic construct
  source 1..24: mol_type = other DNA
tatccatgggtcaccagcagttgg

SEQ ID NO: 2 - DNA, 56 bp, synthetic construct
  source 1..56: mol_type = other DNA
ccaccgccgcttccgccaccgccgcttccaccgccaccactgcagggcacagatgc

SEQ ID NO: 3 - DNA, 55 bp, synthetic construct
  source 1..55: mol_type = other DNA
gtggcggaagcggcggtggcggcagcggcggtggcagcagaaacctccccgtggc

SEQ ID NO: 4 - DNA, 38 bp, synthetic construct
  source 1..38: mol_type = other DNA
gacgcatgcttaggaagcattcagatagctcatcactc

SEQ ID NO: 5 - protein, 544 aa, synthetic construct
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 613 aa, synthetic construct
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASTTTPAPRPPTPAPTIASQPLSLRPEACRPAAGGAVHTRGLDFACDIYIWAPLAGTCGVLLLSLVITLYC

SEQ ID NO: 7 - protein, 613 aa, synthetic construct
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASTTTPAPRPPTPAPTIASQPLSLRPEASRPAAGGAVHTRGLDFASDIYIWAPLAGTCGVLLLSLVITLYC

SEQ ID NO: 8 - protein, 650 aa, synthetic construct
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCV

SEQ ID NO: 9 - protein, 650 aa, synthetic construct
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASVVMRATQLQKNLTSEVWGPTSPKLMLSLKLENKEAKVSKREKAVWVLNPEAGMWQSLLSDSGQVLLESNIKVLPTWSTPVQPMALIVLGGVAGLLLFIGLGIFFCV

SEQ ID NO: 10 - protein, 493 aa, synthetic construct [sequence omitted]

SEQ ID NO: 11 - protein, 492 aa, synthetic construct
MEFGLSWLFLVAILKGVQCSRDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSTRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 45 aa, synthetic construct
TTTPAPRPPTPAPTIASQPLSLRPEASRPAAGGAVHTRGLDFASD

SEQ ID NO: 13 - protein, 82 aa, synthetic construct
VVMRATQLQKNLTSEVWGPTSPKLMLSLKLENKEAKVSKREKAVWVLNPEAGMWQSLLSDSGQVLLESNIKVLPTWSTPVQP

(19) United States
(12) Patent Application Publication
(10) Pub. No.: US 2025/0127897 A1
(43) Pub. Date: Apr. 24, 2025

(54) NATURAL KILLER T-CELLS AND METHODS OF USING THE SAME

(71) Applicant: The University of North Carolina at Chapel Hill, Chapel Hill, NC (US)

(72) Inventors: Gianpietro Dotti, Chapel Hill, NC (US); Elisa Landoni, Chapel Hill, NC (US); Barbara Savoldo, Chapel Hill, NC (US)

(21) Appl. No.: 18/834,037

(22) PCT Filed: Jan. 30, 2023
(86) PCT No.: PCT/US2023/061600

§ 371 Date: Jul. 29, 2024

Related U.S. Application Data
(60) Provisional application No. 63/304,556, filed on Jan. 28, 2022.

(51) Int. Cl. A61K 40/42; A61K 38/20; A61K 40/15; A61K 40/31; A61P 35/00; C07K 14/54; C07K 14/725; C07K 16/30; C12N 15/86

(52) U.S. Cl. CPC A61K 40/4258; A61K 38/208; A61K 40/15; A61K 40/31; A61P 35/00; C07K 14/5434; C07K 14/7051; C07K 16/3084; C12N 15/86; C07K 2317/622; C12N 2740/10043

(57) ABSTRACT

Disclosed herein are modified NKTs expressing human IL-12, compositions comprising the modified NKTs, and therapeutic methods for using the modified NKTs. The modified NKTs described herein are reprogrammed NKTs that demonstrate long term persistence when the expression of IL-12 is coupled with the expression of a CAR. These NKTs acquire long-term capacity to eliminate tumor cells and long-term persistence of NKTs expressing IL-12. Also disclosed herein are modified cells transformed to express a CAR and an exogenous membrane-bound moiety, such as IL-12.

### RELATED APPLICATION(S)

This application claims the benefit of U.S. Provisional Application No. 63/304,556, filed on Jan. 28, 2022. The entire teachings of the above applications are incorporated herein by reference.

### GOVERNMENT SUPPORT

This invention was made with government support under CA243543 awarded by National Institutes of Health (NIH). The government has certain rights in the invention.

### BACKGROUND OF THE INVENTION

Natural killer T cells (NKTs or NKT cells), a specific subset of T cells demonstrated to respond as innate cells and as memory-like cells, bridge innate and adaptive immune responses and have shown promise as a platform for adoptive T-cell therapy in cancer patients. Engineered NKTs expressing chimeric antigen receptors (CARs) represent a class of immunotherapeutics that have shown promising results in preclinical and clinical cancer research and treatment.

NKT CARs can promote indirect antitumor activity through the induction of dendritic cell (DC) maturation and secretion of NK and CD8+ T-cell-activating cytokines. However, many immunosuppressive modifications that malignant cells make to their extracellular microenvironment as well as their own surface protein expression have thus far limited the efficacy and scope of CAR therapies for specific cancers. There remains a need for NKT cells that are modified to possess long-term persistence and capacity to eliminate tumor cells and tumor re-challenge protection.

### SUMMARY OF THE INVENTION

Disclosed herein are modified natural kill T (NKT) cells. In some embodiments, the NKT expresses transgenic IL-12.

In some embodiments, the NKT cell comprises a chimeric antigen receptor (CAR). The CAR may be a GD2.CAR or a CD19.CAR.

In one embodiment, the NKT cell is a human NKT cell. In one embodiment, the NKT cell is a non-human NKT cell, e.g., a mouse NKT cell. In some embodiments, the NKT cell is isolated from peripheral blood.

The modified NKT cells described herein may be produced using any method known to those of skill in the art, including, for example, transduction or transfection. In one embodiment, the NKT cell expressing transgenic IL-12 is produced by transducing an NKT cell with a retroviral supernatant comprising IL12. In one embodiment, the NKT cell expressing transgenic IL-12 is produced using lentiviral vector (LV) transduction. In one embodiment, the NKT cell expressing transgenic IL-12 is produced using mRNA electroporation.

In some embodiments, the transgenic IL-12 is bound to the NKT cell membrane via a CD4 or CD8 stalk. In one embodiment, the transgenic IL-12 is bound to the NKT cell membrane via a CD8 stalk (e.g., a CD8a stalk). The CD8 stalk may comprise a CD8 hinge region and a transmembrane domain. In some embodiments, the CD8 stalk is modified to remove one or more cysteine residues from the hinge region, and in some aspects the CD8 stalk is modified by substituting the one or more cysteine residues with a serine residue. In one embodiment, the CD8 stalk comprises a hinge region with the amino acid sequence of SEQ ID NO: 12. In one embodiment, the transgenic IL-12 is bound to the NKT cell membrane via a CD4 stalk. The CD4 stalk may comprise a CD4 hinge region and a transmembrane domain. In some embodiments, the CD4 stalk is modified to remove one or more cysteine residues from the hinge region, and in some aspects the CD4 stalk is modified by substituting the one or more cysteine residues with a serine residue. In one embodiment, the CD4 stalk comprises a hinge region with the amino acid sequence of SEQ ID NO: 13.

In some embodiments, the NKT cell exhibits one or more features including: promoting enhanced tumor control and improved survival, as compared to a control cell, controlling tumor growth upon tumor re-challenge, as compared to a control cell, increased anti-tumor activity, increased expression of CD62L, long term persistence, enhanced cytotoxic activity upon repetitive exposure to tumor cells in vitro, as compared to a control cell, and/or decreased risk of causing graft versus host disease.

In one embodiment, the NKT cell does not express CD62L.

Also disclosed herein is a population of the modified NKT cells as disclosed herein. Also disclosed herein is a population of genetically modified NKTs isolated from peripheral blood, wherein the NKTs express transgenic IL-12.

In some embodiments, the NKTs of the population comprise a chimeric-antigen receptor (CAR), e.g., a GD2.CAR or a CD19.CAR. In some embodiments, the NKTs of the population are transduced with CAR and IL-12.

In one embodiment, the NKTs do not express CD62L.

Disclosed herein are methods of manufacturing the NKTs disclosed herein by transducing the NKT with a retroviral vector. In some embodiments, the retroviral vector includes IL-12.

In some embodiments, the retroviral vector further includes a green fluorescent protein (GFP). In some embodiments, the retroviral vector further includes an internal ribosomal entry site (IRES). In some embodiments, the retroviral vector further includes a CAR, e.g., a GD2.CAR or a CD19.CAR. In some embodiments, the CAR is a B7H3 CAR or a CSPG4 CAR.

Also disclosed herein are methods of treating cancer by administering to a subject a modified NKT cell as disclosed herein.

In some embodiments, the subject is a mammal, e.g., a human or a mouse. In some embodiments the modified NKT cell is administered to the subject intravenously.

Also disclosed herein is a cell that is transformed to express a CAR and an exogenous membrane-bound moiety, wherein the exogenous membrane-bound moiety comprises a transmembrane domain that is modified to remove one or more cysteine residues that would otherwise be capable of forming a disulphide bond with cysteine residues present in the CAR.

### BRIEF DESCRIPTION OF THE DRAWINGS

FIGS. 1A-1E show NKTs transduced with IL12(i)GFP upregulate CD62L. FIG. 1A is a schematic timeline of the protocol used to select, transduce, and expand NKTs. FIG. 1B shows schematics of the retroviral vectors used to engineer human NKTs. The p40 and p35 subunits of IL-12 are connected by a flexible linker. FIG. 1C shows representative flow cytometry plots of NKT purity in non-transduced NKTs (NT), NKTs transduced with the control GFP vector (GFP) and with the IL12(i)GFP vector. FIG. 1D provides representative flow cytometry plots of NKT transduction efficiency measured as percentage of GFP⁺ cells in non-transduced NKTs (NT), NKTs transduced with the control GFP vector (GFP) and with the IL12(i)GFP vector. FIG. 1E shows representative flow cytometry plots of CD62L in non-transduced NKTs (NT), NKTs transduced with the control GFP vector (GFP) and with the IL12(i)GFP vector.

FIGS. 2A-2E demonstrate NKTs transduced with IL12(i)GFP release IL12 in vitro and persist longer in vivo. FIG. 2A shows quantification of IL-12, IFN-γ, and IL-4 produced by NT, GFP and IL12(i)GFP transduced NKTs in resting condition (unstimulated) or activated with the anti-CD3 anti-CD28 antibodies. Cytokines were measured in supernatants collected 24 hours after plating 1*10⁶ cells/well in 24 well plate in 2 mL of complete media without cytokines. Mean is shown, n=4; *, P=0.0273; **, P=0.0022; ***, P=0.0006; ****, P<0.0001; two-way ANOVA. FIG. 2B provides a volcano plot illustrating the differential gene expression (alpha<0.2) of the RNAseq data between IL12(i)GFP and GFP NKTs. Positive LFC values reflect over-expression in the IL12(i)GFP NKTs. FIG. 2C shows a schematic representation of an in vivo experiment to assess the NKTs persistence in a xenograft NSG mouse model. Mice were engrafted i.v. with 5*10⁶ GFP or IL12(i)GFP Firefly-luciferase labeled NKTs. Mice were imaged with the IVIS kinetic machine at day 0, 2, 4 and 7 post NKTs injection, were euthanized at day 10 and peripheral blood, liver and spleen were collected. FIG. 2D shows representative tumor bioluminescent imaging (BLI) (measured as total flux p/s). FIG. 2E shows quantification of human NKTs (iNKT⁺CD45⁺) in peripheral blood, liver and spleen samples collected 10 days after NKTs infusion. Mean is shown, n=10; *, P=0.0161, **, P=0.0020, ***, P=0.0006, unpaired t test.

FIGS. 3A-3C demonstrate NKTs transduced with the GD2.CAR and IL-12 express the CAR and upregulate the CD62L. FIG. 3A shows schema of the retroviral vectors used to transduce NKTs. For the CD19.CAR the scFv FMC.63 was used and for the GD2.CAR the 1A7 scFv was used. FIG. 3B shows representative flow cytometry plots of CARs expression in control (NT) and CAR-transduced NKTs assessed at day 10 of culture. FIG. 3C shows representative flow cytometry plots of CD62L expression in control (NT) and CAR-transduced NKTs assessed at day 10 of culture.

FIGS. 4A-4D demonstrate GD2.CAR NKTs producing IL-12 have enhanced cytotoxic activity upon repetitive exposure to tumor cells in vitro and mediate long-term tumor control. FIG. 4A shows a summary of the quantification of residual tumor cells after each cycle where NKTs were cocultured with CHLA-225 (E:T=1:1) and then collected and stained with anti-iTCR (Vβ11) and anti-GD2 to identify NKTs and neuroblastoma cells, respectively, by flow cytometry. Mean is shown, n=4. FIG. 4B shows a schematic representation of the metastatic xenograft neuroblastoma model. Mice were engrafted i.v. with 2*10⁶ CHLA-225 Firefly-luciferase labeled neuroblastoma tumor cells. Ten days after, the mice received i.v. 5*10⁶ CAR⁺ NKTs. Mice were imaged with the IVIS kinetic machine weekly, were euthanized at day 80 and peripheral blood, liver and spleen were collected. FIG. 4C shows representative tumor BLI (measured as total flux p/s). FIG. 4D shows representative flow cytometry plots of human NKTs (iNKT⁺CD45⁺) in peripheral blood, liver, and spleen samples collected 80 days after NKTs infusion.

FIGS. 5A-5C demonstrate NKTs transduced with the GD2.CAR and membrane-bound IL-12 express the CAR and upregulate the CD62L. FIG. 5A provides schema of the retroviral vectors used to transduce NKTs. For the GD2.CAR the 1A7 scFv was used. To anchor IL-12 to the membrane of NKTs, a modified CD8a stalk and transmembrane domain were used. FIG. 5B provides representative flow cytometry plots of CAR expression in control CAR-transduced NKTs assessed at day 10 of culture. FIG. 5C provides representative flow cytometry plots of CD62L expression in control CAR-transduced NKTs assessed at day 10 of culture.

FIGS. 6A-6C demonstrate membrane-bound IL-12 is detected on the cell surface and induces the phosphorylation of STAT4. FIG. 6A provides representative flow cytometry plots of IL-12 expression detected on the cell surface of control (NT) or CAR-transduced NKTs assessed at day 10 of culture. FIG. 6B provides representative flow cytometry plots of IL-12 receptor beta (IL-12RB) expression detected on the cell surface of control NT or CAR-transduced NKTs assessed at day 10 of culture. FIG. 6C provides representative western blot illustrating STAT4 phosphorylation in NT, IL12(i)GFP and CAR.GD2(i)IL12TM NKTs at day 14 of culture.

FIGS. 7A-7C demonstrate GD2.CAR NKTs with membrane-bound IL-12 have comparable cytotoxic activity with soluble IL-12 upon repetitive exposure to tumor cells in vitro. FIG. 7A provides representative flow plots of the quantification of residual tumor cells after each cycle where NKTs were cocultured with CHLA-225 (E:T=1:1) and then collected and stained with anti-iTCR (Vβ11) and anti-B7-H3 to identify NKTs and neuroblastoma cells, respectively, by flow cytometry. FIG. 7B shows quantification of IFN-γ and IL-12pr produced by control NKTS (NT) or CAR-transduced NKTs when cocultured with CHLA-225 at E:T ratio 1:1. Cytokines were measured in supernatants collected 24 hours after plating 2.5×10⁵ NKT cells/well with 2.5×10⁵ CHLA-225/well in 24 well plate in 2 mL of complete media without cytokines. Mean and standard deviation are shown, n=2. FIG. 7C shows GD2.CAR(i)IL12TM persistence in vivo. NSG mice were infused with 10*10⁶ GD2.CAR(i)IL12TM NKTs. After 27 days NKTs were detected in the peripheral blood and a high expression of CD62L was maintained.

FIG. 8 provides the amino acid sequences for various IL-12 constructs.

FIG. 9 provides the amino acid sequences for a CD19.CAR and a GD2.CAR.

### DETAILED DESCRIPTION OF THE INVENTION

Natural killer T cells (NKTs or NKT cells) were isolated from peripheral blood and were genetically engineered to express human IL-12. These NKTs were shown to acquire high expression levels of CD62L and to persist long term in immunodeficient mice after adoptive transfer without causing graft versus host disease. Surprisingly, it was discovered that transgenic IL-12 may be used to reprogram and improve NKT cell IL-12 expression rather than merely as soluble cytokines to grow NKT cells. It was further shown that IL-12 by itself is sufficient to support long term engraftment of NKT cells. In some aspects, CD62L may not be needed to support the effects of transgenic IL-12.

Described herein are modified NKT cells, compositions comprising the modified NKT cells, and therapeutic methods for utilizing the modified NKT cells. The modified NKT cells described herein exhibit long term persistence and when the expression of IL-12 is coupled with the expression of a CAR, these NKT cells acquire long-term capacity to eliminate tumor cells and long-term persistence.

### Modified NKTs

Aspects of the disclosure relate to modified T cells, e.g., modified or engineered natural killer T (NKT) cells. In some embodiments, NKT cells are modified to express transgenic IL-12.

In some embodiments, the NKT cells comprise a chimeric antigen receptor (CAR). As is generally known to those of skill in the art, CAR T cells (e.g., CAR NKT cells) may be produced by obtaining NKT cells, such as from a subject in need thereof or from a donor subject and manipulating the cells such that they include chimeric antigen receptors (CARs). The CARs provide the ability to target specific proteins on cancer cells and include an antigen recognition domain, an extracellular hinge region, a transmembrane domain, and an intracellular T cell signaling domain. CAR T cells may be classified as first generation, second generation, third generation, or fourth generation.

First generation CARs are engineered with only the CD3ζ domain. Second generation CARs are engineered with the CD3ζ domain and a costimulatory signaling domain (e.g., CD28 or 4-1BB). Third generation CARs are engineered to include the CD3ζ domain in addition to two costimulatory signaling domains (e.g., both CD28 and CD137). Finally, fourth generation CARs, also referred to as T-cells redirected for universal cytokine-mediated killing (TRUCKs) are engineered to include the CD3ζ domain, two costimulatory signaling domains (e.g., both CD28 and CD137), and some additional genetic modification, such as the addition of transgenes for cytokine secretion or additional costimulatory signaling domains. Any type of CAR may be used in the manufacture of the modified NKT cells. In some embodiments, the CAR is a second generation CAR. In some embodiments, the CAR is selected from the group consisting of a GD2.CAR, a CD19.CAR, B7H3.CAR, and a CSPG4.CAR.

In some embodiments, the NKT cells comprise a T cell receptor (TCR). As is generally known to those of skill in the art, NKT cells comprising a T cell receptor may be produced by obtaining NKT cells, such as from a subject in need thereof or from a donor subject and manipulating the cells such that they include a T cell receptor (TCR). Any type of TCR may be used in the manufacture of the modified NKT cells.

In some embodiments, the NKT cell is a human NKT cell or a non-human NKT cell. In some embodiments, mammalian cells are used. In some embodiments, mammalian cells are primate cells (human cells or non-human primate cells), rodent (e.g., mouse, rat, rabbit, hamster) cells, canine, feline, bovine, or other mammalian cells. In some embodiments, avian cells are used. In some embodiments, the NKT cell is isolated from peripheral blood, bone marrow, lymph, or lymphoid organs. The NKT cell may be isolated by any method known to those of skill in the art.

In some embodiments, the NKT cell is an autologous cell. In some embodiments, the NKT cell is not an autologous cell. In some embodiments, the NKT cell is of the same species of a subject. In some embodiments, the NKT cell is of a species that is different than the species of a subject. In some embodiments, the NKT cells are differentiated from stem or progenitor cells in vitro using differentiation protocols known in the art. In some embodiments, a population of NKT cells is expanded by at least about 10-fold, 20-fold, 30-fold, 40-fold, 50-fold, 60-fold, 70-fold, 80-fold, 90-fold, 100-fold, 200-fold, 500-fold, or greater to provide an increase in the number of NKT cells.

The modified NKT cells described herein exhibit one or more features. Non-limiting examples of the features of the modified NKT cells include promoting enhanced tumor control and improved survival (as compared to a control cell), controls tumor growth upon tumor re-challenge (as compared to a control cell), increased anti-tumor activity, increased expression of CD62L, long term persistence, enhanced cytotoxic activity upon repetitive exposure to tumor cells in vitro (as compared to a control cell), decreased risk of causing graft versus host disease, and combinations thereof. In some embodiments, the modified NKT cell promotes enhanced tumor control and improved survival, as compared to a control cell. In some embodiments, the modified NKT cell controls tumor growth upon tumor re-challenge, as compared to a control cell. In some embodiments, the modified NKT cell exhibits increased anti-tumor activity. In some embodiments, the modified NKT cell exhibits increased expression of CD62L. In some embodiments, the modified NKT cell exhibits long term persistence. In some embodiments, the modified NKT cell exhibits enhanced cytotoxic activity upon repetitive exposure to tumor cells in vitro, as compared to a control cell. In some embodiments, the modified NKT cell exhibits decreased risk of causing graft versus host disease when administer to a subject. In some embodiments, the modified NKT cell does not express CD62L.

In some embodiments, the modified NKT cells contain a membrane-bound cytokine attached to the cell surface (e.g., the cell surface of the NKT cell). In one embodiment, the membrane-bound cytokine is IL-12. In some aspects, the modified NKT cell secretes IL-12, which is then membrane-bound to the surface of the NKT cell. In some embodiments, IL-12 is bound to the cell membrane (e.g., the NKT cell membrane) via a transmembrane domain. In some embodiments, IL-12 is fused to a transmembrane domain. In some embodiments, a transmembrane domain has a sequence that is derived from the transmembrane domain of a molecule selected from the group consisting of an integrin family, CD4, CD8, CD44, glycophorin, MHC Class I and II glycoproteins, EGF receptor, G protein coupled receptor (GPCR) family, receptor tyrosine kinases (e.g., insulin-like growth factor 1 receptor (IGFR) and platelet-derived growth factor receptor (PDGFR)), porin family, other transmembrane proteins known to those of skill in the art, and combinations thereof. In one embodiment, the transmembrane domain comprises a transmembrane domain of CD4. In one embodiment, the transmembrane domain comprises a transmembrane domain of CD8.

In some embodiments, the transgenic IL-12 is bound to the NKT cell membrane via a CD4 or CD8 stalk. In one embodiment, the transgenic IL-12 is bound to the NKT cell membrane via a CD8 stalk. In some embodiments, the CD8 stalk comprises a CD8 hinge region and a transmembrane domain. Suitable CD8 hinge regions and transmembrane domains will be known to those of skill in the art and an illustrative example is provided in SEQ ID NO: 6. In some embodiments, the CD8 stalk is modified to remove one or more cysteine residues from the hinge region. In some embodiments, the CD8 stalk is modified to remove all cysteine residues from the hinge region. In some embodiments, the one or more cysteine residues are removed by substituting the one or more cysteine residues with a serine residue. In some embodiments, the CD8 stalk is modified by substituting all of the cysteine residues of the hinge region to serine residues. In one embodiment, the CD8 stalk is a CD8a stalk. In some embodiments, the CD8 stalk comprises a hinge region with the amino acid sequence of SEQ ID NO: 12, or an amino acid sequence having at least 70%, at least 75%, at least 80%, at least 85%, at least 90%, at least 95%, at least 96%, at least 97%, at least 98%, at least 99% or 100%) sequence identity thereto. In one embodiment, the CD8 stalk comprises, consists, or consists essentially of a hinge region with the amino acid sequence of SEQ ID NO: 12.

In one embodiment, the transgenic IL-12 is bound to the NKT cell membrane via a CD4 stalk. In some embodiments, the CD4 stalk comprises a CD4 hinge region and a transmembrane domain. Suitable CD4 hinge regions and transmembrane domains will be known to those of skill in the art and an illustrative example is provided in SEQ ID NO: 8. In some embodiments, the CD4 stalk is modified to remove one or more cysteine residues from the hinge region. In some embodiments, the CD4 stalk is modified to remove all cysteine residues from the hinge region. In some embodiments, the one or more cysteine residues are removed by substituting the one or more cysteine residues with a serine residue. In some embodiments, the CD4 stalk is modified by substituting all cysteine residues of the hinge region to serine residues. In some embodiments, the CD4 stalk comprises a hinge region with the amino acid sequence of SEQ ID NO: 13, or an amino acid sequence having at least 70%, at least 75%, at least 80%, at least 85%, at least 90%, at least 95%, at least 96%, at least 97%, at least 98%, at least 99% or 100%) sequence identity thereto. In one embodiment, the CD4 stalk comprises, consists, or consists essentially of a hinge region with the amino acid sequence of SEQ ID NO: 13.

In some embodiments, IL-12 is bound to the cell membrane via a CD4 or CD8a stalk. A CD4 or CD8 stalk may include hinge and transmembrane domains. In some aspects, the hinge region (e.g., a CD8 hinge region) is modified to minimize or otherwise prevent its dimerization with the co-expressed CAR. The hinge region may include one or more modifications (e.g., 1, 2, 3, 4, 5, or more amino acid deletions and/or substitutions). In one embodiment, the CD8 hinge region comprises a modification to the cysteine (Cys) residue at position(s) 27 and/or 44 of the CD8a hinge region shown in SEQ ID NO: 6. In one embodiment, the CD8 hinge region comprises a modification to the cysteine (Cys) residue at position(s) C571 and/or C588 of SEQ ID NO: 6. In one embodiment, the CD4 hinge region comprises a modification to the Cys residue at position(s) 14 and/or 56 of the CD4 hinge region shown in SEQ ID NO: 8. In one embodiment, the CD4 hinge region comprises a modification to the Cys residue at position(s) C558 and/or C600 of SEQ ID NO: 8. In some aspects, one or more cysteine residues are substituted for one or more serine (Ser) residues. In one embodiment, the transmembrane domain of the membrane-bound cytokine comprises the sequence TTTPAPRPPTPAPTIASQPLSLRPEASRPAAGGAVHTRGLDFASD (SEQ ID NO: 12). In one embodiment, the transmembrane domain of the membrane-bound cytokine comprises the sequence VVMRATQLQKNLTSEVWGPTSPKLMLSLKLENKEAKVSKREKAVWVLNPE AGMWQSLLSDSGQVLLESNIKVLPTWSTPVQP (SEQ ID NO: 13). It has surprisingly been found that modifying the CD8a stalk to remove the cysteine residues unexpectedly improved the efficacy of the CAR within which the anchored IL-12 was co-expressed.

Without being bound by theory or by a particular mode of application, it is generally understood that cysteine residues present in the membrane-bound moieties (including the native sequences of CD4 or CD8 stalks) likely form disulphide bonds with cysteine residues present in a co-expressed CAR, thereby reducing the efficacy of the CAR. By substituting the cysteine residues that are anticipated to form disulphide bonds between the membrane-bound cytokine (e.g., IL-12) and the CAR, dimerization of the membrane-bound cytokine with the CAR is minimized or otherwise prevented, thereby restoring the efficacy of the CAR. Thus, the present disclosure also extends to a cell that is transformed to express a CAR and an exogenous membrane-bound moiety, wherein the exogenous membrane-bound moiety comprises a transmembrane domain that is modified to remove one or more cysteine residues that would otherwise be capable of forming a disulphide bond with cysteine residues present in the CAR.

In some embodiments, the exogenous membrane-bound moiety is bound to the cell membrane via a CD4 or CD8 stalk. In one embodiment, the membrane-bound therapeutic moiety is bound to the cell membrane via a CD8 stalk. In some aspects, the CD8 stalk comprises a CD8 hinge region and a transmembrane domain. Suitable CD8 hinge regions and transmembrane domains will be known to those of skill in the art and an illustrative example is shown in SEQ ID NO: 6. In some embodiments, the CD8 stalk is modified to remove one or more cysteine residues from the hinge region. In some embodiments, the CD8 stalk is modified to remove all cysteine residues from the hinge region. In some embodiments, the one or more cysteine residues are removed by substituting the one or more cysteine residues with a serine residue. In some embodiments, the CD8 stalk is modified by substituting all cysteine residues of the hinge region to serine residues. In one embodiment, the CD8 stalk is a CD8a stalk. In some embodiments, the CD8 stalk comprises a hinge region with the amino acid sequence of SEQ ID NO: 12, or an amino acid sequence having at least 70%, at least 75%, at least 80%, at least 85%, at least 90%, at least 95%, at least 96%, at least 97%, at least 98%, at least 99%, or 100% sequence identifying thereto. In one embodiment, the CD8 stalk comprises, consists, or consists essentially of a hinge region with the amino acid sequence of SEQ ID NO: 12.

In another embodiment, the exogenous membrane-bound moiety is bound to the cell membrane via a CD4 stalk. In some aspects, the CD4 stalk comprises a CD4 hinge region and a transmembrane domain. Suitable CD4 hinge regions and transmembrane domains will be known to those of skill in the art and an illustrative example is shown in SEQ ID NO: 8. In some embodiments, the CD4 stalk is modified to remove one or more cysteine residues from the hinge region. In some embodiments, the CD4 stalk is modified to remove all cysteine residues from the hinge region. In some embodiments, the one or more cysteine residues are removed by substituting the one or more cysteine residues with a serine residue. In some embodiments, the CD4 stalk is modified by substituting all cysteine residues of the hinge region to serine residues. In some embodiments, the CD4 stalk comprises a hinge region with the amino acid sequence of SEQ ID NO: 13, or an amino acid sequence having at least 70%, at least 75%, at least 80%, at least 85%, at least 90%, at least 95%, at least 96%, at least 97%, at least 98%, at least 99%, or 100% sequence identifying thereto. In one embodiment, the CD4 stalk comprises, consists, or consists essentially of a hinge region with the amino acid sequence of SEQ ID NO: 13.

The present disclosure also extends to a cell that is transformed to express a CAR and an exogenous membrane-bound moiety, wherein the amino acid sequence of the CAR is modified to remove one or more cysteine residues that would otherwise be capable of forming a disulphide bond with cysteine residues present in the transmembrane domain of the exogenous membrane-bound moiety.

In some embodiments, the exogenous membrane-bound moiety is a therapeutic moiety. Suitable therapeutic moieties will be known to those of skill in the art, illustrative embodiments of which include cytokines, an integrin family member, CD4, CD8, CD44, glycophorin, MHC Class I and II glycoproteins, EGF receptor, a G protein coupled receptor (GPCR) family member, a receptor tyrosine kinase (e.g., insulin-like growth factor 1 receptor (IGFR) and platelet-derived growth factor receptor (PDGFR)), a porin family member, and a combination of any of the foregoing. In one embodiment, the therapeutic moiety is a cytokine.

In some embodiments, the cell is an immune cell. Suitable immune cells are known to those of skill in the art and include T cells. In some aspects, the immune cell is a T cell or an NKT cell.

In another embodiment, one or more of the cysteine residues of the membrane-bound CAR that are anticipated to form disulphide bonds with cysteine residues present in the hinge and/or transmembrane regions of the membrane-bound cytokine (e.g., IL-12) may be modified (e.g., substituted for a serine residue), thereby minimizing or otherwise preventing the dimerization of the membrane-bound cytokine with the CAR.

In some embodiments, IL-12 fused to a transmembrane domain comprises a sequence selected from the group consisting of SEQ ID NO: 6, SEQ ID NO: 7, SEQ ID NO: 8, and SEQ ID NO: 9. In some embodiments, IL-12 fused to a CD8 transmembrane domain comprises the sequence SEQ ID NO: 6 or SEQ ID NO: 7. In some embodiments, IL-12 fused to a CD4 transmembrane domain comprises the sequence SEQ ID NO: 8 or SEQ ID NO: 9. In some embodiments, IL-12 fused to a mutated transmembrane domain comprises the sequence SEQ ID NO: 7 or SEQ ID NO: 9. In one embodiment, IL-12 fused to a transmembrane domain comprises the sequence SEQ ID NO: 6. In one embodiment, IL-12 fused to a transmembrane domain comprises the sequence SEQ ID NO: 7. In one embodiment, IL-12 fused to a transmembrane domain comprises the sequence SEQ ID NO: 8. In one embodiment, IL-12 fused to a transmembrane domain comprises the sequence SEQ ID NO: 9.

In some embodiments, the membrane bound or anchored IL-12 contains a linker. Linkers are generally known to those of skill in the art. In some aspects, a linker includes one or more amino acids. For example, a linker may comprise 1, 2, 3, 4, 5, 6, 7, 8, 9, 10, or more amino acids. In some aspects, the linker may be a flexible linker.

NKT cells may be modified, e.g., to express IL-12, using any method known to those of skill in the art. In some embodiments, modifications to the NKT cells may be introduced by RNA targeting agents, such as RNAi, miRNA, or ribozyme. In some cases, the modifications may be introduced via gene editing systems or components thereof. Examples of gene editing systems include CRISPR-Cas systems, zinc finger nuclease (ZFN) systems, TALE systems, TALEN systems, and meganuclease systems. In some embodiments, a gene editing system may be delivered using a vector delivery system. In some embodiments, a gene editing system may be delivered using a viral vector delivery system. In some embodiments, modifications to the NKT cells may be introduced using transduction or transfection. Non-limiting examples of methods for modifying NKT cells include viral, non-viral, and hybrid (viral and non-viral) based methods. Transduction methods may include the use of lentiviral, adenoviral, oncoretroviral, and/or other vectors known to those of skill in the art. Transfection methods may include lipofection, nucleofection, biolistics, virosomes, liposomes, immunoliposomes, polycation or lipid:nucleid acid conjugates, electroporation, sonoporation, magnetofection, gene microinjection, laser irradiation, and other methods known to those of skill in the art.

In some embodiment, an NKT cell expressing transgenic IL-12 is produced by transducing an NKT cell with a viral vector comprising IL-12, e.g., the NKT cell may be transduced with a retroviral supernatant comprising IL-12. In one embodiment, the NKT cell expressing transgenic IL-12 is produced using lentiviral vector (LV) transduction. In one embodiment, the NKT cell expressing transgenic IL-12 is produced using mRNA electroporation.

In some embodiments, the NKT cell is modified to reduce or knock out expression of CD62L. In some embodiments, the NKT cells are modified using a gene editing system, such as a CRISPR-associated system, e.g., CRISPR/Cas9. In some embodiments, the NKT cell is modified using CRISPR/Cas to reduce or knock out expression of one or more targets. In some embodiments, the NKT cell is modified using CRISPR/Cas to reduce or knock out expression of CD62L.

In some embodiments, NKTs cell are isolated from a mammal and genetically modified (i.e., transduced or transfected in vitro) with IL-12, and in some embodiments with a CAR. In some embodiments, a NKT cell can be transduced with a viral vector (e.g., a retroviral vector) or transfect with a plasmid or nucleic acid construct. In some embodiments, the retroviral vector includes IL-12. The IL-12 may have p40 and p35 subunits connected via a flexible linker. In some embodiments, the retroviral vector further includes SFG and/or GFP. In some embodiments, the retroviral vector further includes an internal ribosomal entry site (IRES). In some embodiments, the NKT cell is transduced with a CAR and IL-12.

For administration to a subject, modified NKT cells produced by the methods as disclosed herein can be administered to a subject, for example in pharmaceutically acceptable compositions. These pharmaceutically acceptable compositions comprise a therapeutically-effective amount of modified NKT cells as described above, formulated together with one or more pharmaceutically acceptable carriers (additives) and/or diluents. In some embodiments the pharmaceutical compositions further include diluents and/or other components, and/or other cytokines and/or cell populations.

As described herein, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. Nos. 3,773,919; and 35 3,270,960. In some embodiments, direct administration to a tumor and/or a body cavity, orifice, and/or tissue containing a tumor may be desired.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Methods of Treatment

Disclosed herein are methods of treating or preventing a cancer in a subject in need thereof. In some embodiments, the method includes administering a modified NKT cell expressing transgenic IL-12, and in some embodiments comprising a chimeric antigen receptor (CAR), as described herein. In some embodiments the method includes administering a therapeutically effective amount of modified NKT cells expressing transgenic IL-12, and in some embodiments comprising a CAR.

Also disclosed herein are methods of generating and/or expanding a population of modified NKT cells in a subject (e.g., a subject diagnosed with cancer and/or otherwise in need thereof). In some embodiments, the method includes administering to a subject a NKT cell expressing transgenic IL-12. In some aspects, the population of modified NKT cells persists in the subject for a period of time following administration to the subject (e.g., at least one week, one month, two months, three months, four months, five months, six months, nine months, one year, two years, five years, etc.).

In some embodiments, the cells described herein, e.g., modified NKT cells are transplantable, e.g., modified NKT cells can be administered to a subject. In some embodiments, the subject who is administered modified NKT cells is the same subject from whom the pre-modified NKT cells was obtained (e.g., for autologous cell therapy). In some embodiments, the subject is a different subject. In some embodiments, a subject is suffering from cancer, or is a normal subject. For example, the modified NKT cells for transplantation can be a form suitable for transplantation.

The method can further include administering the modified NKT cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In some aspects, the source of the cells is the peripheral blood of a subject.

A composition comprising modified NKT cells can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

As used herein, the term “administer” refers to the placement of a composition into a subject by a method or route which results in at least partial localization of the composition at a desired site such that desired effect is produced. Routes of administration suitable for the methods of the invention include both local and systemic administration. Generally, local administration results in more of the administered modified NKT cells being delivered to a specific location as compared to the entire body of the subject, whereas, systemic administration results in delivery of the modified NKT cells to essentially the entire body of the subject.

In the context of administering modified NKT cells, the term “administering” also include transplantation of such cells in a subject. As used herein, the term “transplantation” refers to the process of implanting or transferring at least one cell to a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species). A skilled artisan is well aware of methods for implanting or transplantation of cells for treating cancer, which are amenable to the present invention.

Modified NKT cells or compositions comprising the same can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. In addition, the methods and compositions described herein can be used to treat domesticated animals and/or pets.

In some embodiments a subject is deemed “at risk” of having or developing cancer or recurrence of cancer. Whether a subject is at risk of having or developing cancer or having a recurrence of cancer is a determination that may be within the discretion of the skilled practitioner caring for the subject. Any suitable diagnostic test and/or criteria can be used. For example, a subject may be considered “at risk” of having or developing cancer if (i) the subject has a mutation, genetic polymorphism, gene or protein expression profile, and/or presence of particular substances in the blood, associated with increased risk of developing or having cancer relative to other members of the general population not having mutation or genetic polymorphism; (ii) the subject has one or more risk factors such as having a family history of cancer, having been exposed to a carcinogen or tumor-promoting agent or condition, e.g., asbestos, tobacco smoke, aflatoxin, radiation, chronic infection/inflammation, etc., advanced age; (iii) the subject has one or more symptoms of cancer, (iv) the subject has a medical condition that is known to increase the likelihood of cancer, etc.

As used herein, the type of cancer is not limited. The term “cancer” as used herein is defined as a hyperproliferation of cells whose unique trait—loss of normal controls—results in unregulated growth, lack of differentiation, local tissue invasion, and metastasis. With respect to the inventive methods, the cancer can be any cancer, including any of acute lymphocytic cancer, acute myeloid leukemia, adenocarcinoma, alveolar rhabdomyosarcoma, anal cancer, angiosarcoma, B cell lymphoma, basal cell carcinoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, colorectal cancer, esophageal cancer, cervical cancer, endometrial cancer, fibrosarcoma, gastrointestinal carcinoid tumor, hematopoietic neoplasias, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, sarcoma, skin cancer, small intestine cancer, soft tissue cancer, solid tumors, squamous cell carcinoma, stomach cancer, T cell lymphoma, testicular cancer, thymoma, thyroid cancer, ureter cancer, urinary bladder cancer, and uterine cancer. As used herein, the term “tumor” refers to an abnormal growth of cells or tissues of the malignant type, unless otherwise specifically indicated and does not include a benign type tissue.

As used herein, the term “treating” and “treatment” refers to administering to a subject an effective amount of modified NKT cells altered ex vivo according to the methods described herein so that the subject has a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a disorder associated with expression of a polynucleotide sequence, as well as those likely to develop such a disorder due to genetic susceptibility or other factors.

By “treatment,” “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

The dosage, administration schedule and method of administering the modified NKT cells are not limited. The dosage will depend upon a variety of factors including other treatment, the number of doses and the individual patient parameters including age, physical condition, size and weight. These are factors well known to those of ordinary skill in the art and can be addressed with no more than routine experimentation. In some embodiments, a maximum tolerated dose may be used, that is, the highest safe and tolerable dose according to sound medical judgment. In some embodiments, a pharmaceutical composition comprising the modified NKT cells can be administered at a dosage of about 10³ to about 10¹⁰ cells/kg body weight, and in some embodiments, the dosage can be from about 10⁵ to about 10⁶ cells/kg body weight, including all integer values (e.g., 10⁴, 10⁵, 10⁶, 10⁷, 10⁸, 10⁹) within those ranges.

The dose used may be the maximal tolerated dose or a sub-therapeutic dose or any dose therebetween. In some embodiments modified NKT cells are administered in combination with one or more agents. In some embodiments, the modified NKT cells and/or the one or more agents are administered according to a defined administration schedule. Multiple doses are contemplated. In some embodiments, when the modified NKT cells and one or more agents are administered in combination, a sub-therapeutic dosage of one or more of the agents may be used. A “sub-therapeutic dose” as used herein refers to a dosage which is less than that dosage which would produce a therapeutic result in the subject if administered in the absence of the other agent. In some aspects, a sub-therapeutic dose of an anticancer agent is one which would not produce a useful therapeutic result in the subject in the absence of the administration of the modified NKT cells described herein. Therapeutic doses of anticancer agents are well known in the field of medicine for the treatment of cancer.

As used herein, pharmaceutical compositions comprise one or more agents or compositions that have therapeutic utility, and a pharmaceutically acceptable carrier, e.g., a carrier that facilitates delivery of agents or compositions. Agents and pharmaceutical compositions disclosed herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or as an aerosol. Depending upon the type of condition (e.g., cancer) to be treated, compounds of the invention may, for example, be inhaled, ingested or administered by systemic routes. Thus, a variety of administration modes, or routes, are available. The particular mode selected will typically depend on factors such as the particular compound selected, the particular condition being treated and the dosage required for therapeutic efficacy. The methods described herein, generally speaking, may be practiced using any mode of administration that is medically acceptable, meaning any mode that produces acceptable levels of efficacy without causing clinically unacceptable adverse effects. Preferred modes of administration are parenteral and oral routes. The term “parenteral” includes subcutaneous, intravenous, intramuscular, intraperitoneal, and intrasternal injection, or infusion techniques. In some embodiments, inhaled medications are of particular use because of the direct delivery to the lung, for example in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. In some embodiments agents are delivered by pulmonary aerosol. Other appropriate routes will be apparent to one of ordinary skill in the art.

Toxicity and therapeutic efficacy of administration of compositions comprising modified NKT cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising modified NKT cells that exhibit large therapeutic indices are preferred.

The amount of a composition comprising modified NKT cells can be tested using several well-established animal models.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising modified NKT cells can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of NKT cells. In one embodiment of the invention, an isolated population of modified NKT cells as disclosed herein may be used for the production of a pharmaceutical composition, for the use in transplantation into subjects in need of treatment, e.g. a subject that has, or is at risk of developing cancer. Examples include subjects with melanoma or pancreatic cancer. In some embodiments, an isolated population of modified NKT cells as disclosed herein may be autologous and/or allogeneic. In some embodiments, the subject is a mammal, and in other embodiments the mammal is a human.

One embodiment of the invention relates to a method of treating cancer in a subject comprising administering an effective amount of a composition comprising modified NKT cells as disclosed herein to a subject with cancer. Other embodiments relate to a method of treating a tumor in a subject comprising administering an effective amount of a composition comprising modified NKT cells as disclosed herein to a subject with a tumor.

In some embodiments, the modified NKT cells as disclosed herein are administered to a subject having cancer in combination with a second therapeutic treatment (e.g., chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytotoxin, fludaribine, cyclosporin, FK506, rapamycin, mycophenolic acid, steroids, FR901228, cytokines, and/or irradiation).

In some embodiments, the modified NKT cells are administered to a patient in conjunction with (e.g., before, concurrently and/or following) bone marrow transplantation, T cell ablative therapy using either chemotherapy agents such as fludarabine, external-beam radiation therapy (XRT), cyclophosphamide, or antibodies such as OKT3 or CAMPATH. In another embodiment, the modified NKTs are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects can receive an infusion of the expanded modified NKT cells. In an additional embodiment, expanded cells can be administered before and/or following surgery.

In the treatment of cancers or tumors the modified NKT cells may optionally be administered in conjunction with other, different, cytotoxic agents such as chemotherapeutic or antineoplastic compounds or radiation therapy useful in the treatment of the disorders or conditions described herein (e.g., chemotherapeutics or antineoplastic compounds). The other compounds may be administered prior to, concurrently and/or after administration of the modified NKT cells. As used herein, the word “concurrently” means sufficiently close in time to produce a combined effect (that is, concurrently may be simultaneously, or it may be two or more administrations occurring before or after each other)

As used herein, the phrase “radiation therapy” includes, but is not limited to, x-rays or gamma rays which are delivered from either an externally applied source such as a beam or by implantation of small radioactive sources.

Nonlimiting examples of suitable chemotherapeutic agents which may be administered with the modified NKT cells as described herein include daunomycin, cisplatin, verapamil, cytosine arabinoside, aminopterin, democolcine, tamoxifen, Actinomycin D, Alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine, Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), Mithramycin, Deoxyco-formycin, Mitomycin-C, L-Asparaginase, Interferons (especially IFN-a), Etoposide, and Teniposide; Other anti-proliferative cytotoxic agents are navelbene, CPT-11, anastrazole, letrazole, capecitabine, reloxafine, cyclophosphamide, ifosamide, and droloxafine. Additional anti-proliferative cytotoxic agents include, but are not limited to, melphalan, hexamethyl melamine, thiotepa, cytarabin, idatrexate, trimetrexate, dacarbazine, L-asparaginase, camptothecin, topotecan, bicalutamide, flutamide, leuprolide, pyridobenzoindole derivatives, interferons, and interleukins. Preferred classes of antiproliferative cytotoxic agents are the EGFR inhibitors, Her-2 inhibitors, CDK inhibitors, and Herceptin® (trastuzumab). (see, e.g., U.S. Pat. Nos. 6,537,988; 6,420,377). Such compounds may be given in accordance with techniques currently known for the administration thereof.

In some embodiments, the modified NKT cells as disclosed herein may be administered in any physiologically acceptable excipient, where the modified NKT cells may find an appropriate site for replication, proliferation, and/or engraftment. In some embodiments, the modified NKT cells as disclosed herein can be introduced by injection, catheter, or the like. In some embodiments, the modified NKT cells as disclosed herein can be frozen at liquid nitrogen temperatures and stored for long periods of time, being capable of use on thawing. If frozen, the modified NKT cells will usually be stored in a 10% DMSO, 50% FCS, 40% RPMI 1640 medium. Once thawed, the cells may be expanded by use of growth factors and/or feeder cells associated with culturing NKT cells.

In some embodiments, the modified NKT cells as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising the modified NKT cells as disclosed herein will be adapted in accordance with the route and device used for administration. In some embodiments, a composition comprising the modified NKT cells can also comprise or be accompanied with one or more other ingredients that facilitate the engraftment or functional mobilization of the modified NKT cells. Suitable ingredients include matrix proteins that support or promote adhesion of the modified NKT cells, or complementary cell types. In another embodiment, the composition may comprise resorbable or biodegradable matrix scaffolds.

In some embodiments, the modified NKT cells can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. Modified NKT cells can be administered to a subject at the following locations: clinic, clinical office, emergency department, hospital ward, intensive care unit, operating room, catheterization suites, and radiologic suites.

In other embodiments, the modified NKT cells are stored for later implantation/infusion. The modified NKT cells may be divided into more than one aliquot or unit such that a portion of the modified NKT cells are retained for later application while part is applied immediately to the subject. Moderate to long-term storage of all or part of the cells in a cell bank is also within the scope of this invention, as disclosed in U.S. Patent Publication No. 2003/0054331 and Patent Publication No. WO 03/024215, which are incorporated by reference in their entireties. At the end of processing, the concentrated cells may be loaded into a delivery device, such as a syringe, for placement into the recipient by any means known to one of ordinary skill in the art.

Pharmaceutical compositions comprising effective amounts of NKT cells are also contemplated by the present invention. These compositions comprise an effective number of modified NKT cells, optionally, in combination with a pharmaceutically acceptable carrier, additive or excipient. Systemic administration of modified NKT cells to the subject may be preferred in certain indications, whereas direct administration at the site of or in proximity a tumor may be preferred in other indications.

In some embodiments, modified NKT cells can optionally be packaged in a suitable container with written instructions for a desired purpose, such as the reconstitution or thawing (if frozen) of the modified NKT cells prior to administration to a subject.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

As used herein “A and/or B”, where A and B are different claim terms, generally means at least one of A, B, or both A and B. For example, one sequence which is complementary to and/or hybridizes to another sequence includes (i) one sequence which is complementary to the other sequence even though the one sequence may not necessarily hybridize to the other sequence under all conditions, (ii) one sequence which hybridizes to the other sequence even if the one sequence is not perfectly complementary to the other sequence, and (iii) sequences which are both complementary to and hybridize to the other sequence.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### EXAMPLES

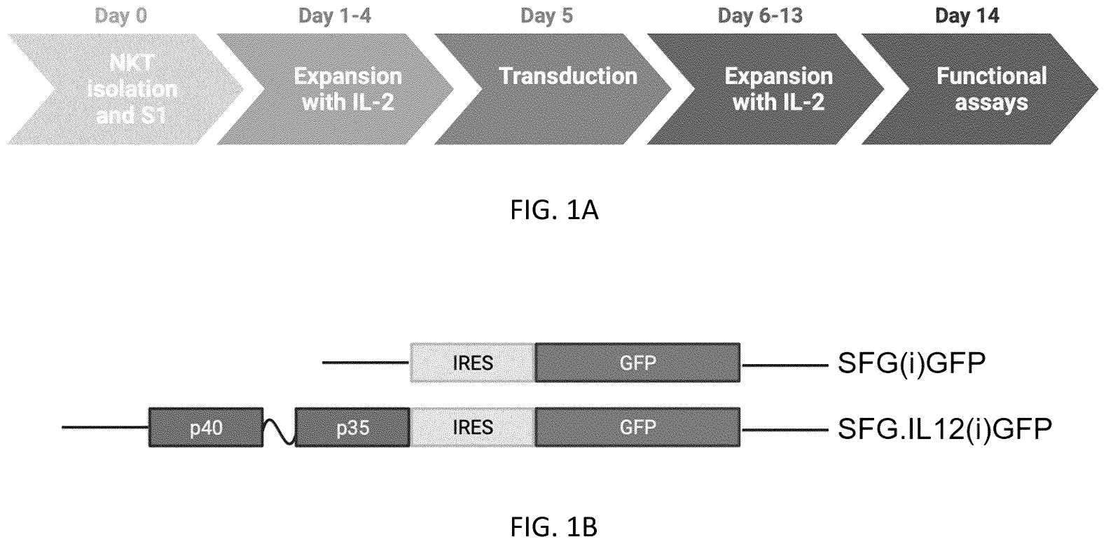

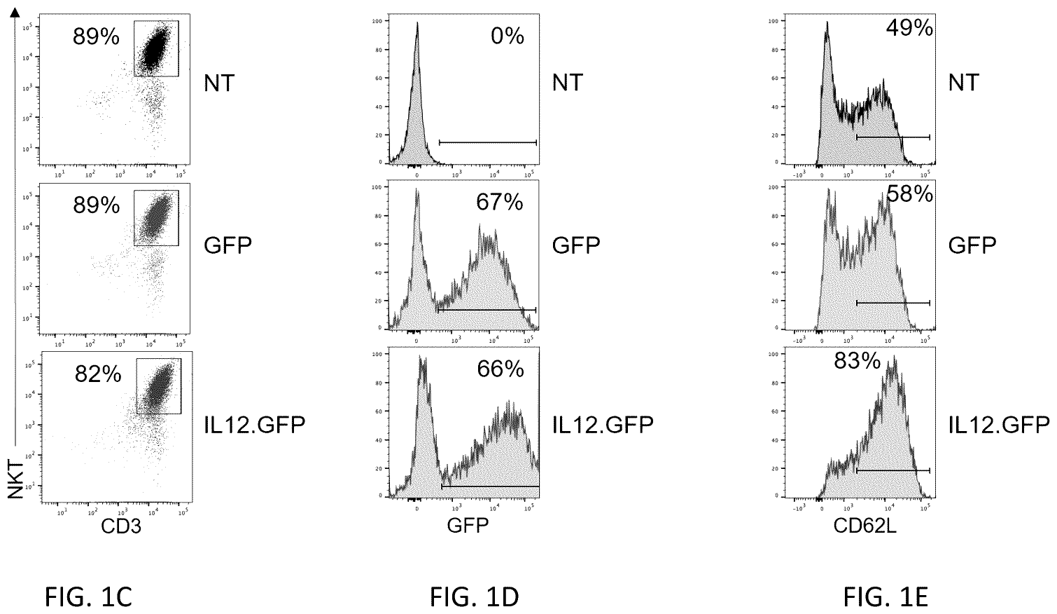

Modified NKT cells are obtained optionally via the method outlined in FIG. 1A: Day 0, NKT cells are isolated; Days 1-4, the population of NKT cells is expanded with IL-2; Day 5, NKT cells are transduced with at least one retroviral vector; Days 6-13, the population of NKT cells is expanded with IL-2; and Day 14, the NKT cells are assessed using one or more functional assays. The transduction of the NKT cells at Day 5 occurs with one or more retroviral vectors (FIG. 1B). One retroviral vector is SFG(i)GFP and a second retroviral vector is SFG.IL12(i)GFP. Both vectors include IRES and GFP. The SFG.IL12(i)GRP vector includes the p40 and p35 subunits of IL-12 connected via a flexible linker.

The modified NKT cells were assessed after being fed with cytokines. Flow cytometry plots showed NKT cell purity, NKT cell transduction efficiency as a percentage of GFP+ cells, and CD62L expression in non-transduced NKT cells (NT), NKT cells transduced with the control GFP vector (GFP), and the NKT cells transduced with the IL12(i)GFP vector (FIGS. 1C-1E). FIG. 1E clearly shows that CD62L is significantly upregulated in NKT cells transduced with IL12(i)GFP.

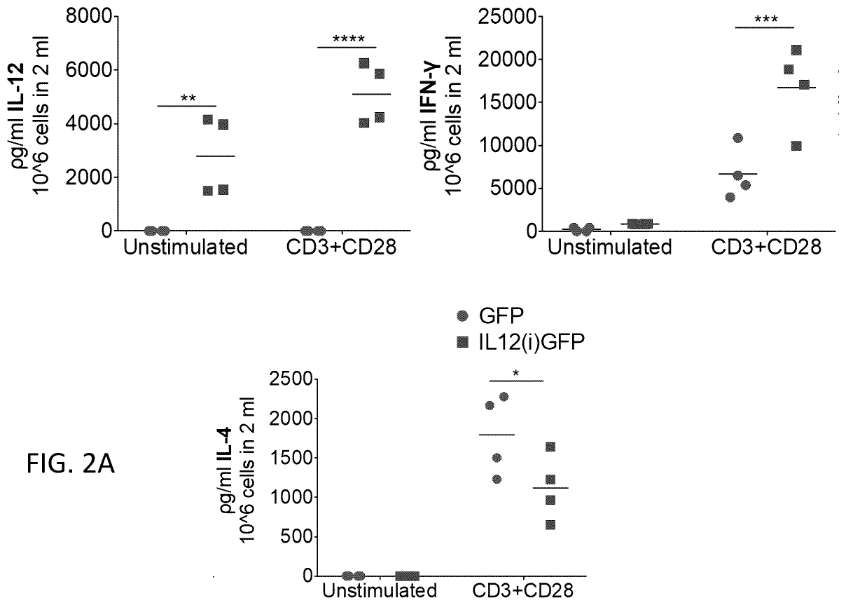

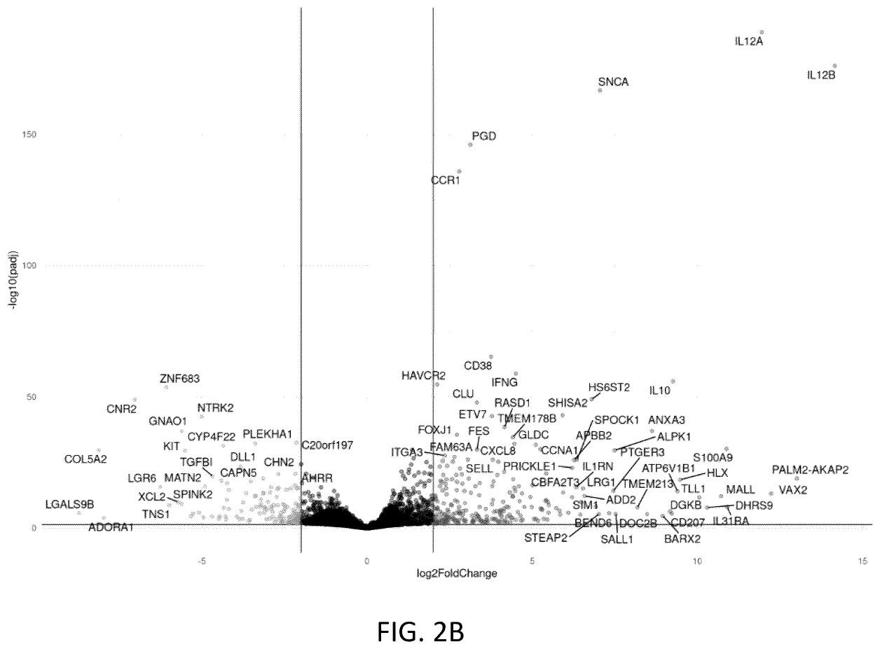

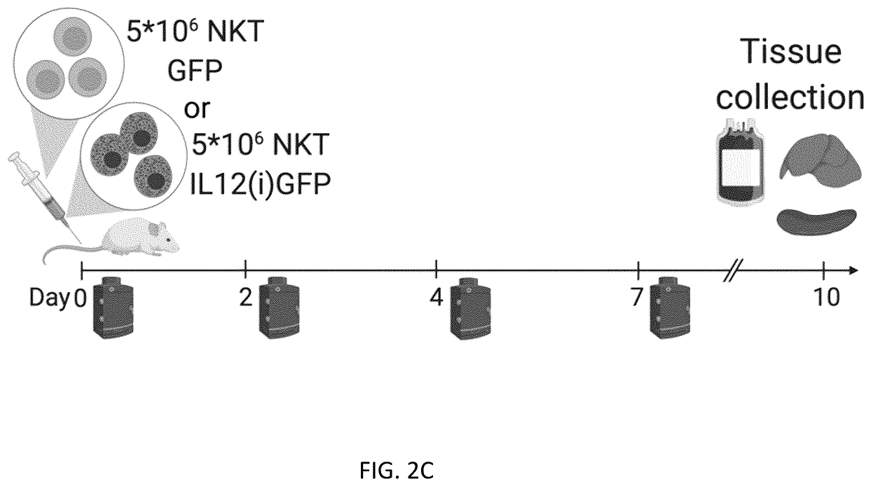

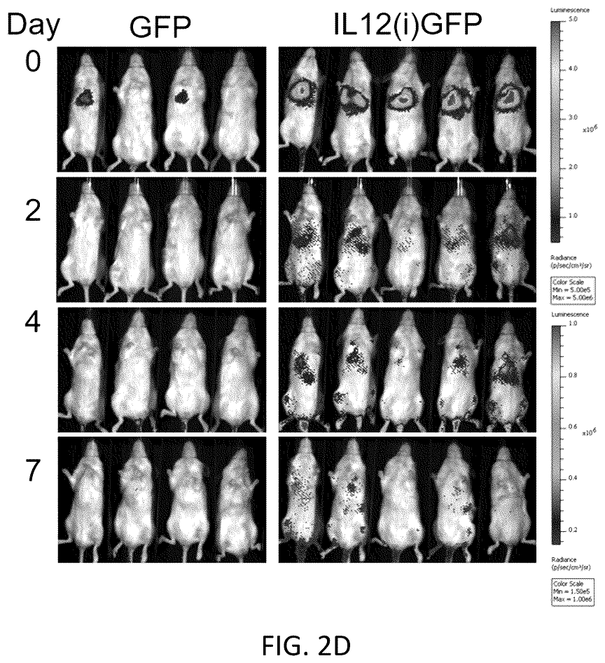

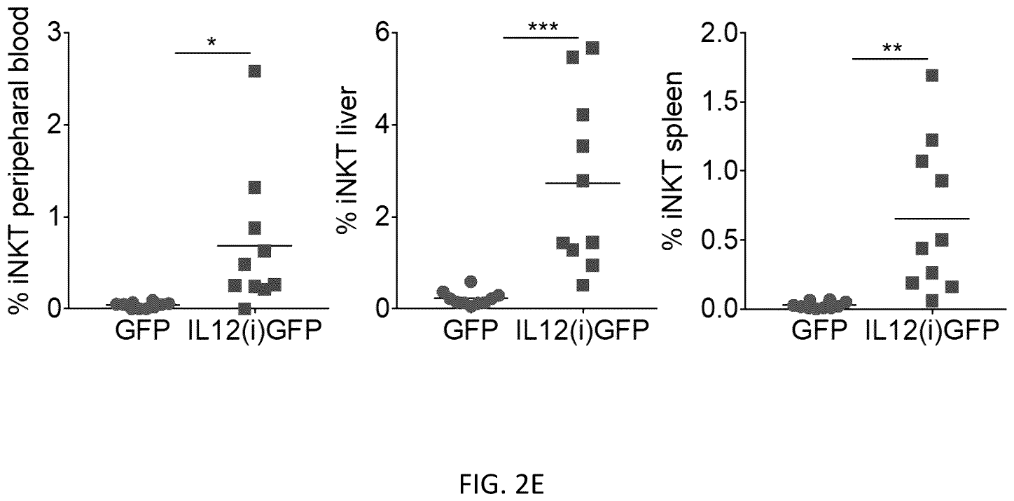

Various cytokines, including IL-12, IFN-γ and IL-4, produced by NKT cells (NT, GFP, and IL12(i)GFP NKT cells) were measured and quantified (FIG. 2A). The NKT cells were either in a resting condition (unstimulated) or were activated (treatment with anti-CD3 and anti-CD28 antibodies). NKT cell persistence was then assessed in a xenograph NSG mouse model where the mice were engrafted with GFP or IL12(i)GFP firefly-luciferase labeled NKT cells (FIG. 2C). The mice were imaged with an IVIS kinetic machine at day 0, 2, 4 and 7 post NKT cell injection and were then euthanized at day 10. The tissue collection included peripheral blood, liver and spleen. The tumor bioluminescent imaging (BLI) was shown for the GFP mice and IL12(i)GFP mice at day 0, 2, 4, and 7 (FIG. 2D). In addition, the quantification of the human NKT cells in the peripheral blood, liver, and spleen was also provided for samples collected 10 days after the NKT cell infusion (FIG. 2E).

To fully characterize the transcriptomic effects of IL-12 in NKTs, IL12(i)GFP and GFP NKTs were compared by performing RNASeq. Increased expression was observed of IL-12A/B introduced by the retroviral vector in the IL12(i)GFP NKTs. Additionally, differential expression of approximately 380 genes was observed between IL12(i)GFP and GFP NKTs (FIG. 2B) including HAVRC2, SELL, and IFNG, as well as proinflammatory genes. In addition, high expression of CD62L was identified compared to GFP NKTs, which is consistent with human NKTs with high proliferative potential in vivo. Moreover, IL-12 NKTs acquired a pro-inflammatory phenotype since, after activation through the iTCR or the CAR, they produced more IFN-g and less IL-4 compared to control NKTs.

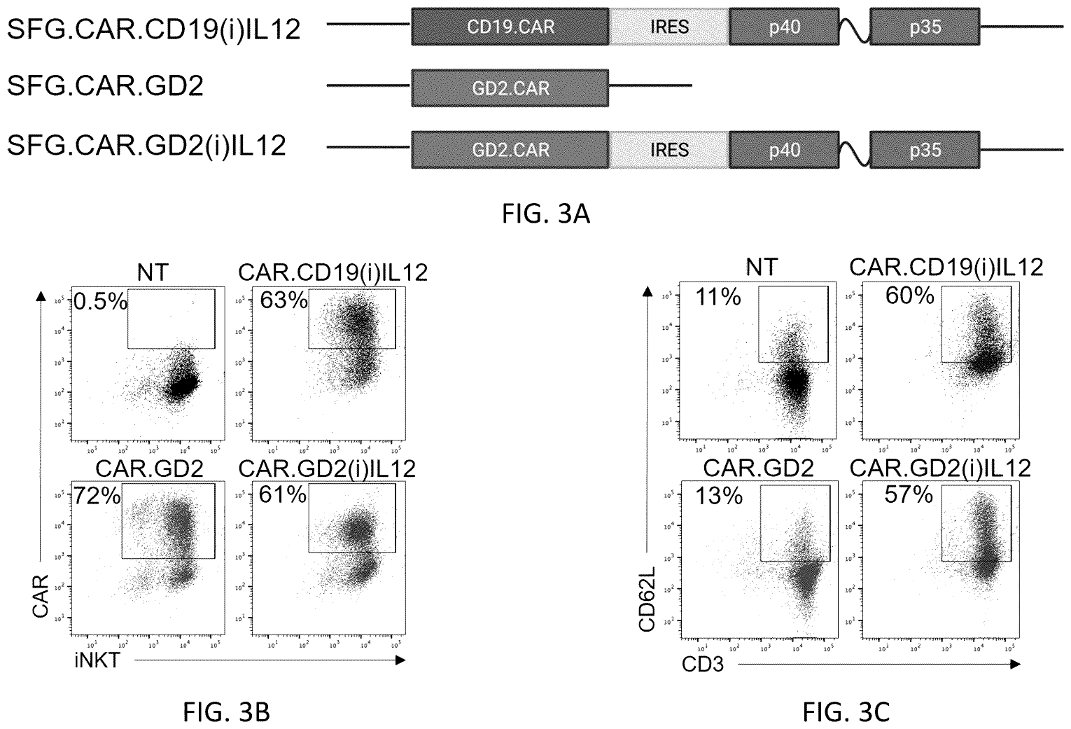

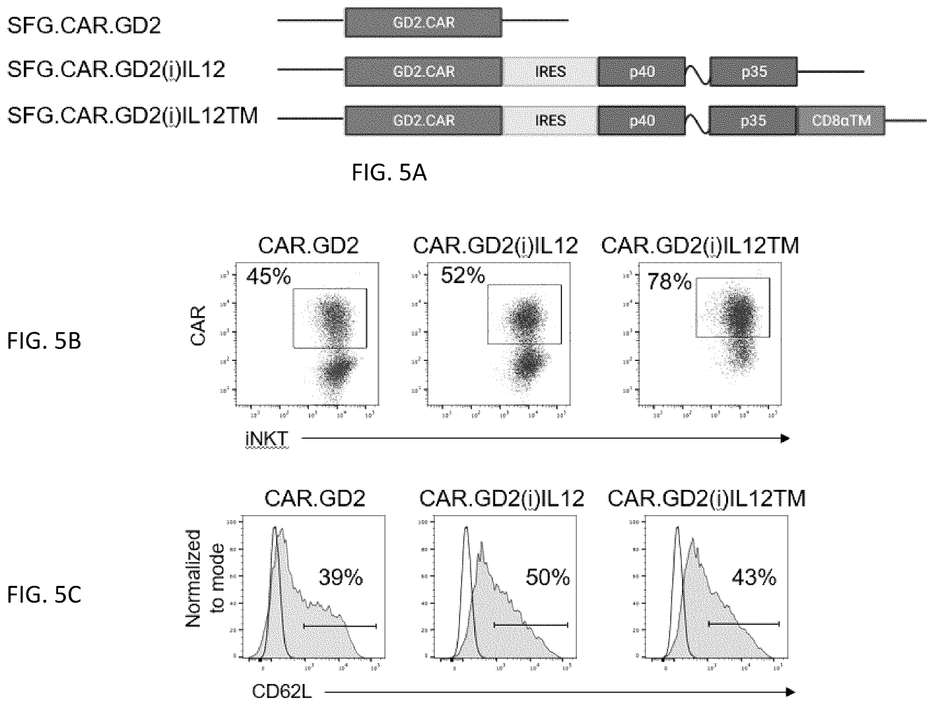

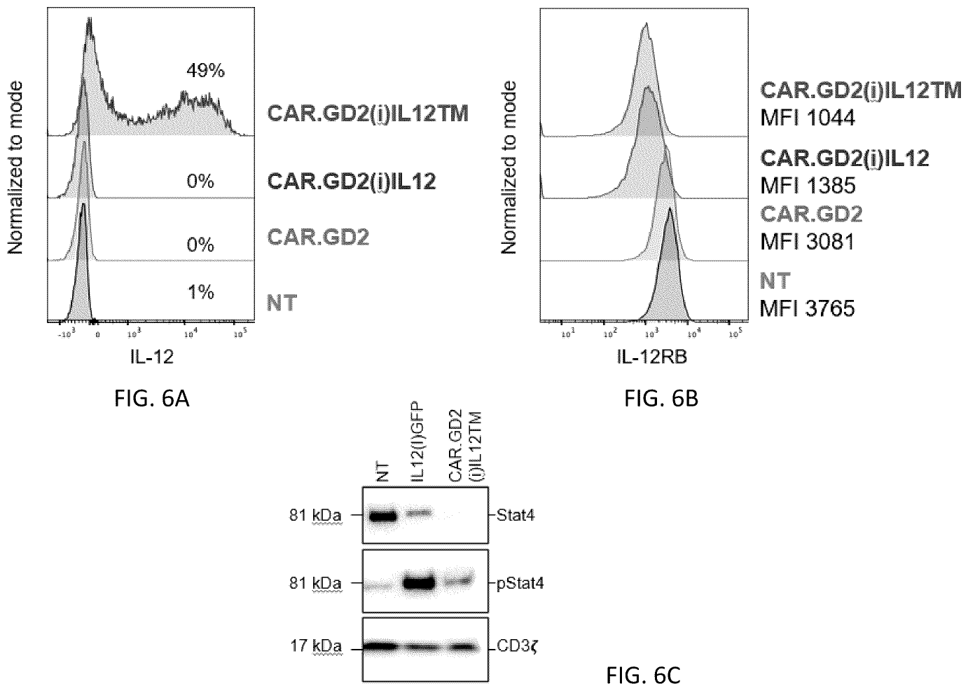

Various retroviral vectors were assessed for the transduction of NKT cells with a CAR. A first retroviral vector was SFG.CAR.CD19(i)IL12; a second retroviral vector was SFG.CAR.GD2; a third retroviral vector was SFG.CAR.GD2(i)IL12; and a fourth retroviral vector was SFG.CAR.GD2(i)IL12TM (FIG. 3A and FIG. 5A). The CD19.CAR used scFv FMC.63 and the GD2.CAR used 1A7 scFv. Representative flow cytometry plots showed the CARs expression, as well as CD62L expression, in control (NT) and CAR-transduced NKT cells assessed at day 10 of culture (FIGS. 3B-3C and FIGS. 5B-5C).

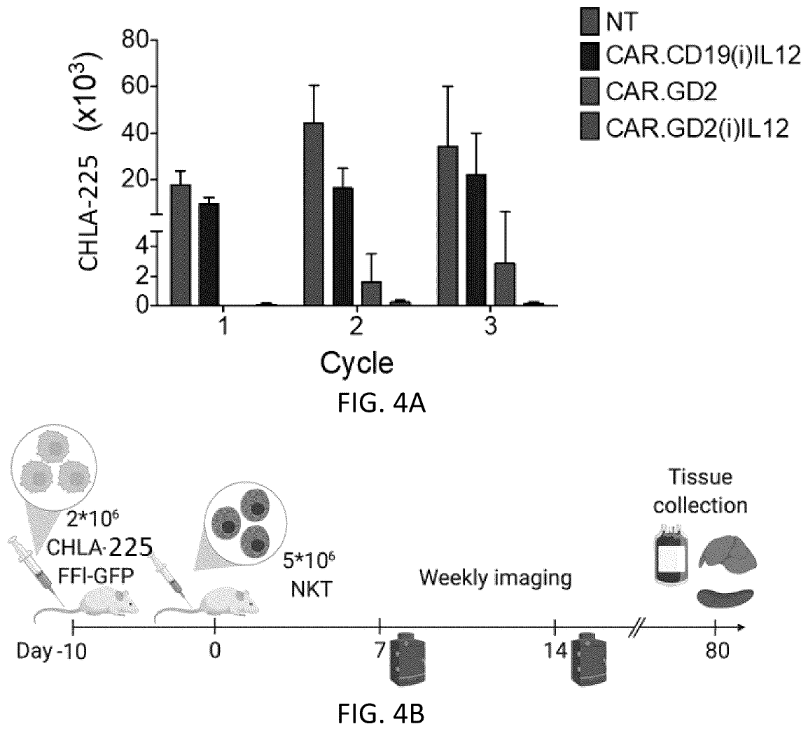

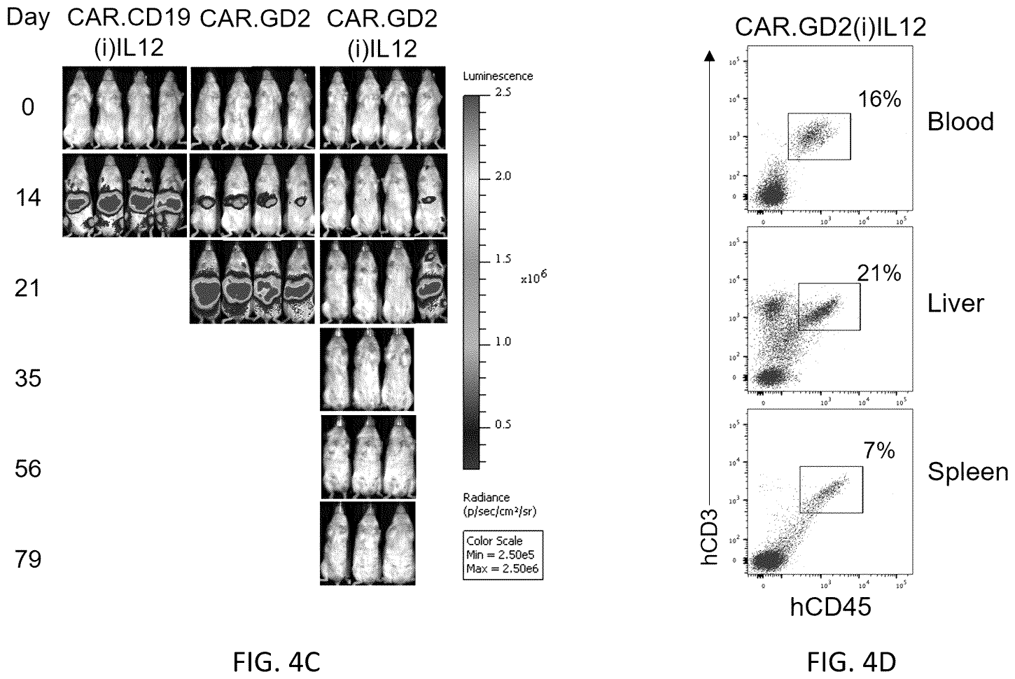

NKT cells transduced with the three CAR retroviral vectors discussed above from FIG. 3A were cocultured with CHLA-225 (E:T=1:1). Residual tumor cells were collected and stained with anti-iTCR (Vβ11) and anti-GD2 to identify and quantify NKT cells and neuroblastoma cells, respectively, by flow cytometry (FIG. 4A). Mice were engrafted intravenously with 2*10⁶ CHLA-225 Firefly-luciferase labeled neuroblastoma tumor cells. Ten days after, the mice were intravenously given 5*10⁶ CAR+ NKT cells (as described in FIG. 3A). The mice were imaged with the IVIS kinetic machine weekly and euthanized at day 80. Samples were collected from the peripheral blood, liver, and spleen (FIG. 4B). In addition, tumor BLI was provided at Day 0, 14, 21, 35, 56, and 79 (FIG. 4C) and the amount of human NKT cells (iNKT⁺CD45⁺) in the peripheral blood, liver, and spleen was measured and plotted (FIG. 4D).

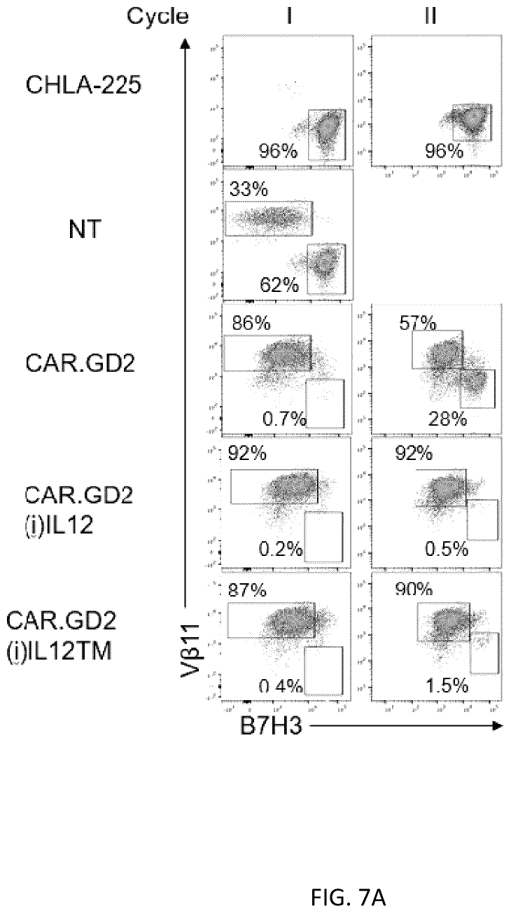

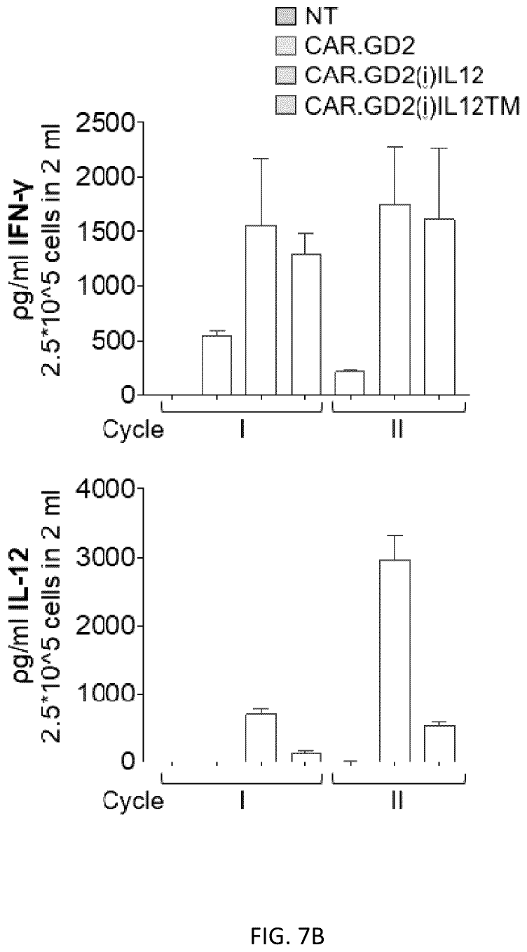

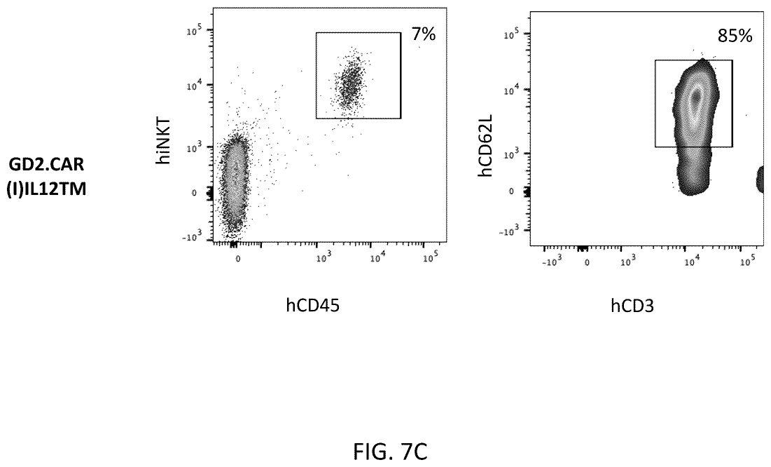

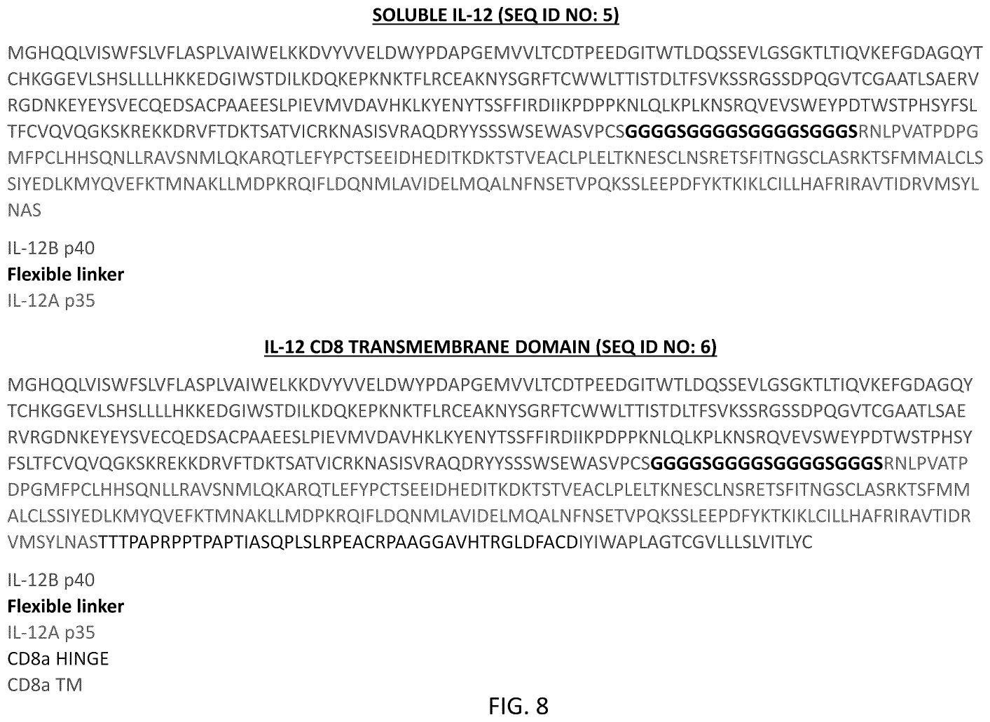

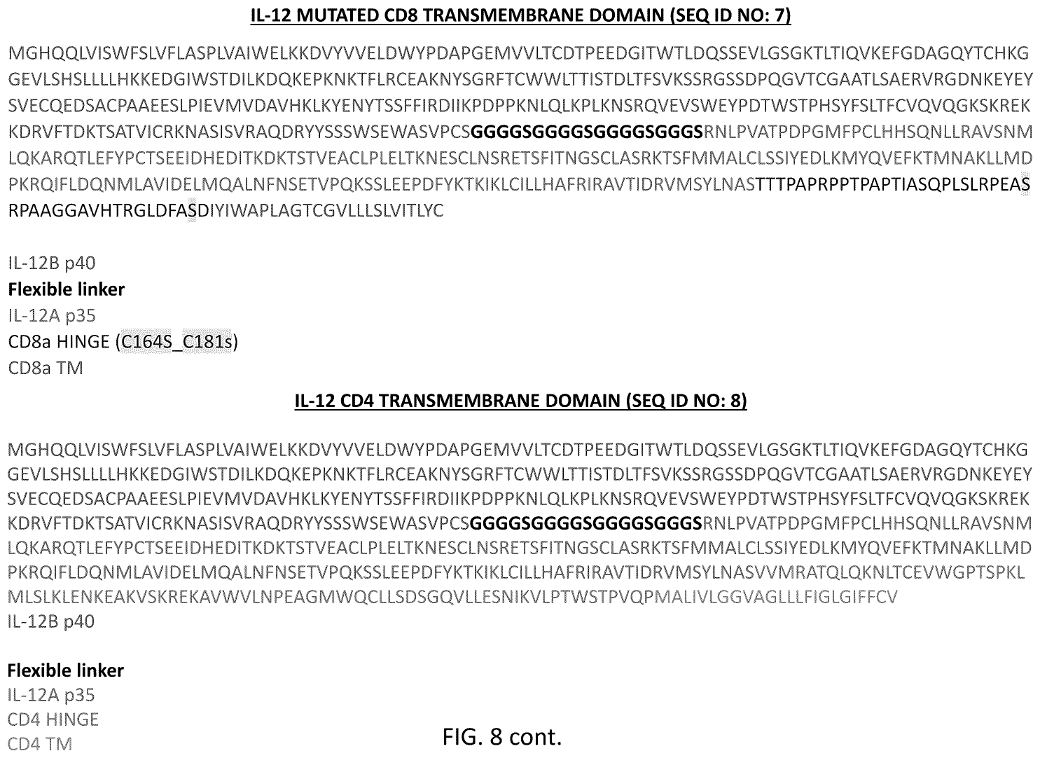

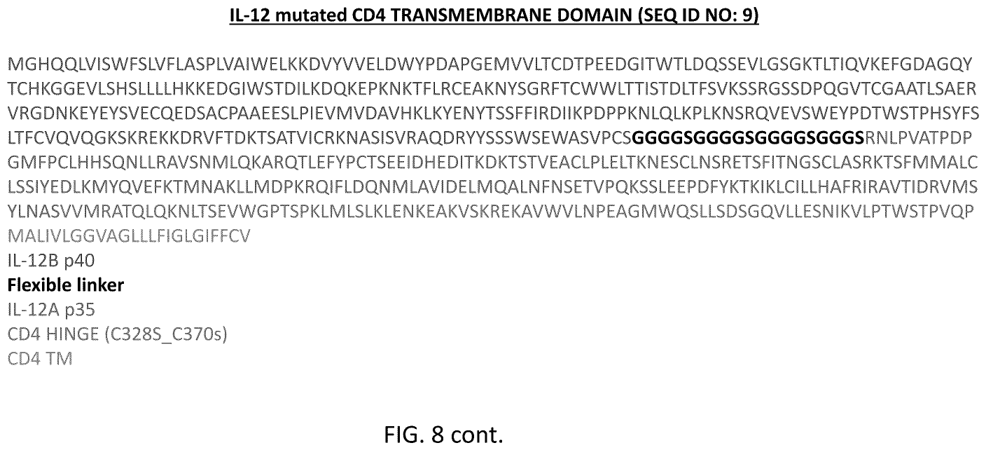

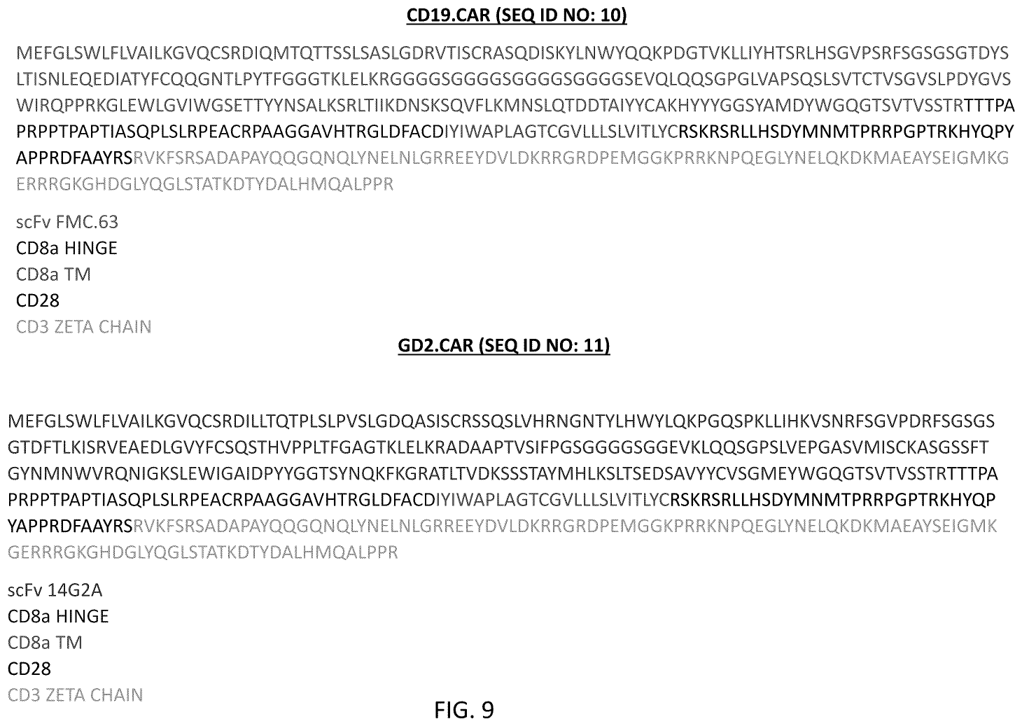

NKT cells transduced with the three CAR retroviral vectors discussed above from FIG. 5A. AS shown in FIG. 5B, NKT cells transduced with a retroviral vector encoding a GD2 CAR and the membrane-bound IL-12 unexpectedly showed a greater level of GD2 CAR expression (SFG.CAR.GD2(i)IL12TM; 78%) when compared to NKT cells that were transduced with the retroviral vector encoding a GD2 CAR and soluble IL-12 (SFG.CAR.GD2(i)IL12; 52%). The NKT cells transduced with the three CAR retroviral vectors discussed above from FIG. 5A were cocultured with CHLA-225 (E:T=1:1). Residual tumor cells were collected and stained with anti-iTCR (Vβ11) and anti-GD2 to identify and quantify NKT cells and neuroblastoma cells, respectively, by flow cytometry. As shown in FIG. 7A, upon repetitive exposure to tumor cells in vitro, GD2.CAR NKTs with membrane-bound IL-12 showed comparable cytotoxic activity with GD2.CAR NKTs that express soluble IL-12. While IFN-γ production by GD2.CAR NKTs was greater than control NKTs (NT), there was no significant difference in the level of IFN-γ production between GD2.CAR NKTs with membrane-bound IL-12 and GD2.CAR NKTs with soluble IL-12 (FIG. 7B). As expected, GD2.CAR NKTs modified to express soluble IL-12 showed greater IL-12 production when compared to the NT and GD2.CAR NKTs with membrane-bound IL-12 (FIG. 7B). As shown in FIG. 7C, NKTs transduced with the GD2.CAR(i)IL12TM retroviral vector showed persistence in vivo. Mice will be engrafted intravenously with CHLA-225 Firefly-luciferase labeled neuroblastoma tumor cells. Ten days later, the mice will be intravenously given CAR+ NKT cells (as described in FIG. 5A). The mice will be imaged with an IVIS kinetic machine weekly and euthanized at day 80. Samples will be collected from the peripheral blood, liver, and spleen. In addition, tumor BLI will be provided at various points of time and the amount of human NKT cells (iNKT⁺CD45⁺) in the peripheral blood, liver, and spleen will be measured and plotted.

A future mouse model will include the use of the NKT cells transduced with the CAR retroviral vectors, as described above and in FIG. 3A and FIG. 5A, and the NKT cells will be further modified to reduce or eliminate expression of CD62L. The expression of CD62L may be reduced or eliminated using any technique known to those of skill in the art.

Based on the data from the experiments above, it was surprisingly discovered that IL-12 reprograms NKTs for long term persistence. In addition, when NKT cells were transduced to co-express a CAR, the NKTs unexpectedly acquire long-term capacity to eliminate tumor cells and protect mice from tumor re-challenge, further indicating the long-term persistence of NKTs expressing IL-12.

### Methods

Cell lines. The tumor cell line CHLA-225 was maintained in culture with RPMI 1640 (Gibco) supplemented with 10% FBS (Sigma), 1% L-glutamine (Gibco), and 1% penicillin-streptomycin (Gibco) in a humidified atmosphere containing 5% CO2 at 37° C. The CHLA-225 cell line was transduced with a SFG gamma retroviral vector encoding the firefly luciferase gene (FFluc). Cells were kept in culture for less than 2 consecutive months, after which aliquots from the original expanded vial were used. All tumor cell lines were routinely tested to exclude contamination with mycoplasma and assessed for the expression of tumor markers by flow cytometry to confirm identity.

Retroviral constructs and generation of retroviral supernatants. Retroviral supernatants were prepared by transient transfection of 293T cells and used to transduce NKTs (isolated as stated below). The sequence of the p40 and p35 subunits of IL-12 were obtained from the NCBI website and were linked together with a flexible linker described from Anderson R. et al. through overlapping PCR

(Forward p40: TATCCATGGGTCACCAGCAGTTGG (SEQ ID
NO: 1); Reverse p40:
CCACCGCCGCTTCCGCCACCGCCGCTTCCACCGCCACCACTGCAGGGCA
CAGATGC (SEQ ID NO: 2); Forward p35:
GTGGCGGAAGCGGCGGTGGCGGCAGCGGCGGTGGCAGCAGAAACCTCCC
CGTGGC (SEQ ID NO 3); Reverse p35:
GACGCATGCTTAGGAAGCATTCAGATAGCTCATCACTC (SEQ ID
NO: 4)).

The whole IL-12 cassette was cloned into the SFG retroviral vector containing the IRES and GFP. As control the vector SFG containing the IRES and GFP only was used. The cassette encoding the GD2-specific scFv (scFv14G2a), the CD8a stalk and transmembrane domain, the CD28 intracellular domain and CD3z chain was previously cloned into the SFG backbone (GD2.CAR). The IL-12 was then cloned in the GD2.CAR cassette separated by the IRES element (GD2.CAR(i)IL12). Finally, for selected experiments, the scFv14G2a in the GD2.CAR(i)IL12 vector was swapped with the CD19-specific scFv (scFv FMC.63) to obtain the CD19.CAR(i)IL12 vector.

To generate the GD2.CAR(i)IL12TM construct the stop codon was removed at the end of the p35 subunit from GD2.CAR(i)IL12 and the CD8a stalk and transmembrane domain was added.

NKT cell isolation, transduction and in vitro expansion. Buffy coats from healthy volunteer blood donors were purchased from the Gulf Coast Regional Blood Center (Houston, Texas, USA). Peripheral blood mononuclear cells (PBMCs) were isolated by Lymphoprep (Accurate Chemical and Scientific Corporation) density gradient centrifugation. NKTs were purified from PBMCs using anti-iNKT microbeads (Miltenyi Biotech). NKTs were cultured in complete medium, consisting of 45% Click's medium (Irvine Scientific), 45% RPMI 1640 (Hyclone), 10% FBS (Hyclone), 1% L-glutammine (Gibco) and 1% penicillin-streptomycin (Gibco). Upon NKTs selection, the negative fraction was used as feeders, after irradiation (40 Gy) at the PBMCs:NKTs ratio of 10:1 in the presence of α-galactosylceramide (αGalCer, 100 ng/ml Diagnocine LLC) and IL-2 (200 IU/mL Stem Cell Factor). NKTs were transduced in retronectin coated plates on day 5 and further expanded for 10 days in the presence of IL-2 and then used for functional assays.

Immunophenotyping: NKT cells were stained with antibodies (Ab) against CD3 (APC-H7, clone SK7), CD62L (BV605, clone DREG-56), CD4 (PE-Cy7, clone SK3), CD8 (Alexa Fluor 700, clone RPA-T8) and CD45 (APC) from BD Biosciences and IL-12 (p70, APC) and CD212 (IL12R b2, APC) from Miltenyi Biotech. Tumor cells were stained with Abs against GD2 (PE) and CD276 (B7-H3, BV421, clone 7-517) from BD Biosciences. The purity of NKTs was assessed by staining the cells with the PE-conjugated Ab specific for TCR Va24 chain (anti-iNKT, clone 6B11, from BD Biosciences) or for TCR β11 chain (FITC, Beckman Coulter). To detect the GD2.CAR expression in transduced NKT cells, the 1A7 anti-idiotype mAb specific for the 14G2a.scFv and PE or APC-conjugated rat anti-mouse secondary mAbs were used (BD Biosciences). Data acquisition was performed on a BD FACSCanto II or BD LSRFortessa using the BD FACS-Diva software. Data analyses were performed with the FlowJo software.

Western blot: Protein lysate was resolved on 4%-15% SDS polyacrylamide gel electrophoresis gels (SDS-PAGE, Bio-Rad). After protein transfer onto Polyvinylidene fluoride membranes (Bio-Rad), membranes were blocked in 5% non-fat milk in TBS-T and incubated with primary and secondary Abs in TBS-T with 1% milk. The following Abs were used: α-Stat4 (C46B10, dilution 1:1000) and α-Phospho-Stat4 (Tyr693, dilution 1:1000) from Cell Signaling; α-CD3ζ (dilution 1:1000) horseradish peroxidase (HRP) conjugated from Santa Cruz; HRP conjugated secondary Ab(Goat-α-Rabbit #32460, dilution 1:500) from Thermo Scientific. Incubation with the primaries Abs was done overnight at 4° C. while the incubation with the secondary Ab was done 1 hour at R.T. Membranes were developed either with Clarity Max Western ECL Substarte (Bio-Rad) or with SuperSignal West Femto Maximum Sensitivity Substrate (Thermo Scientific) on a Gel station (Bio-Rad).

ELISA: NKTs (5*10⁵ cells/well) were cultured in 24-well plates coated with anti-CD3 (1 mg/mL, Miltenyi Biotech) and anti-CD28 (1 mg/mL, BD Biosciences). Wells non coated were used as negative control. Supernatants were harvested after 24 hours of culture from each cycle of coculture. IFN-γ, IL-4 and IL-12 were measured using a specific ELISA (R&D System).

Repetitive coculture: NKTs (2.5*10⁵ cells/well) were co-cultured with CHLA-225 at an E:T ratio of 1:1 in 24-well plates, in the absence of cytokines (Cycle I). At day 3, all cells were transferred into a new well pre-seeded with 2.5*10⁵ neuroblastoma cells (Cycle II). At day 6, all cells were transferred into a new well pre-seeded with 2.5*10⁵ neuroblastoma cells (Cycle III). At the end of every cycle, cells were harvested and stained for CD3 or TCR β11 and GD2 or B7-H3 mAbs to detect NKTs and tumor cells, respectively. The number of residual tumor cells in culture was enumerated by flow cytometry using CountBright absolute counting beads (Invitrogen).

Xenogenic neuroblastoma model: In the experiment to assess the persistence of NKTs in vivo, female and male NSG mice (7-9 weeks of age, obtained from the UNC Animal Core) were injected intravenously (i.v.) via tail injection with 5*10⁶ FFluc-labeled GFP or IL12(i)GFP NKTs. NKTs persistence was monitored for 7 days by bioluminescence (BLI; total flux, photons/second) using the IVIS kinetic in vivo imaging system (PerkinElmer). Mice were euthanized at day 10 and peripheral blood was collected from the heart and spleen, liver were smashed on cell strainers and washed with 2 mL of PBS. Peripheral blood, spleen and liver were analyzed to detect the presence of NKTs [stained with Abs against CD3 (APC-H7, clone SK7) and CD45 (APC, clone 2D1)] by flow cytometry to enumerate the percentage of human NKTs. In the experiment to assess the anti-tumor activity of NKTs in vivo, female and male NSG mice (7-9 weeks of age, obtained from the UNC Animal Core) were injected intravenously (i.v.) via tail injection with 2*10⁶ FFluc-labeled CHLA-225 tumor cells. Seven days after tumor cell injection, mice were infused i.v. with 5*10⁶ CAR.CD19(i)IL12, CAR.GD2 or CAR.GD2(i)IL12 transduced NKTs. Neuroblastoma tumor growth was monitored weekly by bioluminescence (BLI; total flux, photons/second) using the IVIS kinetic in vivo imaging system (PerkinElmer). Mice were sacrificed according to pre-determined guidelines for tumor growth and signs of discomfort or to terminate the experiment. When mice were euthanized peripheral blood was collected from the heart and spleen, liver were smashed on cell strainers and washed with 2 mL of PBS. Peripheral blood, spleen and liver were analyzed to detect the presence of NKTs [stained with Abs against CD3 (APC-H7, clone SK7) and CD45 (APC, clone 2D1)] by flow cytometry to enumerate the percentage of human NKTs.

What is claimed is:

1. A modified natural killer T (NKT) cell, wherein the NKT cell expresses transgenic IL-12.

2. The modified NKT cell of claim 1, comprising a chimeric antigen receptor (CAR).

3. The modified NKT cell of claim 1, wherein the CAR is a GD2.CAR.

4. The modified NKT cell of claim 1, wherein the CAR is a CD19.CAR.

5. The modified NKT cell of claim 1, wherein the NKT cell is a human NKT cell.

6. The modified NKT cell of claim 1, wherein the NKT cell is a non-human NKT cell.

7. The modified NKT cell of claim 1, wherein the NKT cell is a mouse NKT cell.

8. The modified NKT cell of claim 1, wherein the NKT cell is isolated from peripheral blood.

9. The modified NKT cell of claim 1, wherein the NKT cell expressing transgenic IL-12 is produced by transducing an NKT cell with a retroviral supernatant comprising IL12.

10. The modified NKT cell of claim 1, wherein the transgenic IL-12 is bound to the NKT cell membrane.

11. The modified NKT cell of claim 10, wherein the transgenic IL-12 is bound to the NKT cell membrane via a CD4 or CD8 stalk.

12. The modified NKT cell of claim 10, wherein the transgenic IL-12 is bound to the NKT cell membrane via a CD8 stalk.

13. The modified NKT cell of claim 12, wherein the CD8 stalk comprises a CD8 hinge region and a transmembrane domain.

14. The modified NKT cell of claim 13, wherein the CD8 stalk is modified to remove one or more cysteine residues from the hinge region.

15. The modified NKT cell of claim 13, wherein the CD8 stalk is modified to substitute one or more cysteine residues from the hinge region with a serine residue.

16. The modified NKT cell of any one of claims 12 to 15, wherein the CD8 stalk is a CD8a stalk.

17. The modified NKT cell of claim 16, wherein the CD8 stalk comprises a hinge region having the amino acid sequence of SEQ ID NO: 12.

18. The modified NKT cell of claim 10, wherein the transgenic IL-12 is bound to the NKT cell membrane via a CD4 stalk.

19. The modified NKT cell of claim 18, wherein the CD4 stalk comprises a CD4 hinge region and a transmembrane domain.

20. The modified NKT cell of claim 19, wherein the CD4 stalk is modified to remove one or more cysteine residues from the hinge region.

21. The modified NKT cell of claim 19, wherein the CD4 stalk is modified to substitute one or more cysteine residues from the hinge region with a serine residue.

22. The modified NKT cell of claim 18, wherein the CD4 stalk comprises a hinge region having the amino acid sequence of SEQ ID NO: 13.

23. The modified NKT cell of claim 1, wherein the NKT cell promotes enhanced tumor control and improved survival, as compared to a control cell.

24. The modified NKT cell of claim 1, wherein the NKT cell controls tumor growth upon tumor re-challenge, as compared to a control cell.

25. The modified NKT cell of claim 1, wherein the NKT cell exhibits increased anti-tumor activity.

26. The modified NKT cell of claim 1, wherein the NKT cell exhibits increased expression of CD62L.

27. The modified NKT cell of claim 1, wherein the NKT cell exhibits long term persistence.

28. The modified NKT cell of claim 1, wherein the NKT cell exhibits enhanced cytotoxic activity upon repetitive exposure to tumor cells in vitro, as compared to a control cell.

29. The modified NKT cell of claim 1, wherein the NKT cell exhibits decreased risk of causing graft versus host disease.

30. The modified NKT cell of claim 1, wherein the NKT cell does not express CD62L.

31. A population of the modified NKT cells of claim 1.

32. A population of genetically modified natural killer T cells (NKTs) isolated from peripheral blood wherein the NKTs express transgenic IL-12.

33. The population of claim 32, wherein the NKTs comprise a chimeric-antigen receptor (CAR).

34. The population of claim 33, wherein the CAR is GD2.CAR.

35. The population of claim 33, wherein the CAR is CD19.CAR.

36. The population of claim 32, wherein the NKTs are transduced with CAR and IL-12

37. The population of claim 32, wherein the NKTs do not express CD62L.

38. A method of manufacturing the modified NKT cells of claim 1, comprising transducing the NKT cell with a retroviral vector.

39. The method of claim 38, wherein the retroviral vector comprises IL-12.

40. The method of claim 38, wherein the retroviral vector comprises a green fluorescent protein (GFP).

41. The method of claim 38, wherein the retroviral vector comprises an internal ribosomal entry site (IRES).

42. The method of claim 38, wherein the retroviral vector comprises a CAR.

43. The method of claim 38, wherein the retroviral vector comprises CD19.CAR.

44. The method of claim 38, wherein the retroviral vector comprises GD2.CAR.

45. A method of treating cancer in a subject comprising administering to the subject a therapeutically effective amount of a modified NKT cell of claim 1.

46. The method of claim 45, wherein the subject is a mammal.

47. The method of claim 45, wherein the subject is human.

48. The method of claim 45, wherein the modified NKT cell is administered to the subject intravenously.

49. A cell transformed to express a CAR and an exogenous membrane-bound moiety, wherein the exogenous membrane-bound moiety comprises a transmembrane domain modified to remove one or more cysteine residues.

50. The cell of claim 49, wherein the one or more cysteine residues removed would otherwise be capable of forming a disulphide bond with cysteine residues present in the CAR.

51. The cell of claim 49, wherein the exogenous membrane-bound moiety is IL-12.

52. The cell of claim 49, wherein the exogenous membrane-bound moiety is bound to a cell membrane via a CD4 or CD8 stalk.

53. The cell of claim 49, wherein the transmembrane domain comprises a CD8 stalk.

54. The cell of claim 53, wherein the CD8 stalk comprises a CD8 hinge region.

55. The cell of claim 49, wherein the transmembrane domain is modified to substitute the one or more cysteine residues with a serine residue.

56. The cell of claim 49, wherein the transmembrane domain comprises a CD8a hinge region having the amino acid sequence of SEQ ID NO: 12.

57. The cell of claim 49, wherein the exogenous membrane-bound moiety is bound to a cell membrane via a CD4 stalk.

58. The cell of claim 57, wherein the CD4 stalk comprises a CD4 hinge region.

59. The cell of claim 49, wherein the transmembrane domain comprises a CD4 hinge region having the amino acid sequence of SEQ ID NO: 13.
